(19) United States
(12) Patent Application Publication
(10) Pub. No.: US 2023/0228734 A1
(43) Pub. Date: Jul. 20, 2023

(54) METHOD FOR FIBRINOGEN MEASUREMENT

(71) Applicant: A&T CORPORATION, Fujisawa-shi, Kanagawa (JP)

(72) Inventor: Kazuya NAKAMOTO, Kanagawa (JP)

(21) Appl. No.: 18/000,608

(22) PCT Filed: Apr. 28, 2021
(86) PCT No.: PCT/JP2021/017035

§ 371 Date: Dec. 2, 2022

(30) Foreign Application Priority Data
Jun. 5, 2020 (JP) JP 2020-098512

(51) Int. Cl. G01N 33/49; G01N 33/86

(52) U.S. Cl. CPC G01N 33/4905; G01N 33/86; G01N 2333/75; G01N 2333/974

(57) ABSTRACT

This invention provides a method that enables determining the fibrinogen concentration in plasma of a sample. The method comprises: computing the fibrinogen concentration in whole blood of the sample using magnetic particles; computing the waveform-based hematocrit value based on the peak value of the movement signal of the magnetic particles; subjecting the fibrinogen concentration in whole blood to hematocrit correction using the waveform-based hematocrit value; and computing the fibrinogen concentration in plasma of the sample.

### TECHNICAL FIELD

The present invention relates to a method for fibrinogen measurement.

### BACKGROUND ART

Fibrinogen plays a key role in the blood coagulation cascade and hemostasis. Determination of fibrinogen concentration is a test intended to examine the normality/abnormality in blood clotting ability along with prothrombin time (also referred to as “PT”) and activated partial thromboplastin time (also referred to as “APTT”), and is extensively performed in clinical practice and, in particular, in clinical laboratories.

Examples of techniques that can readily determine fibrinogen concentration by adding a sample to a dry reagent card dropwise include the fibrinogen measurement dry reagent disclosed in Patent Literature 1 and the fibrinogen determination method disclosed in Patent Literature 2. The fibrinogen measurement dry reagent disclosed in Patent Literature 1 involves the use of diluted blood plasma. The method disclosed in Patent Literature 2 requires sample preparation and a whole blood sample is diluted to 7.5- to 10-fold, a plasma sample is diluted to 15-fold, and then the sample is added to a reagent card dropwise. However, when fibrinogen is to be analyzed urgently in, for example, a delivery room or operating room or at bed side, it is difficult to use a system that requires a dilution procedure as an essential procedure.

On the other hand, examples of techniques that enable fibrinogen determination with an undiluted sample include the method disclosed in Patent Literature 3. According to the method disclosed in Patent Literature 3, use of an undiluted sample involves the use of an excessive amount of thrombin, so as to convert all fibrinogens into fibrin monomers. Further, in order to suppress the polymerization reaction of the resulting fibrin monomers and prolong the clotting time, a fibrin monomer polymerization inhibitor (G-P-R-P-A-amide) is used. According to the method disclosed in Patent Literature 3, it is necessary to dissolve reagents in purified water in advance to prepare a liquid reagent and incubate the reagent until immediately before the measurement. In addition, calibration is necessary before the measurement. That is, it is difficult to apply the method disclosed in Patent Literature 3 to urgent fibrinogen determination because of the need of incubation of the dissolved reagent or calibration. The technique disclosed in Patent Literature 3 does not adopt a dry reagent card system. Further, and in general, a composition suitable for a reagent to be reacted in a liquid state is different from a composition suitable for a dry reagent card.

In recent years, importance of fibrinogen determination has been pointed out in the perioperative period and perinatal period. In the case of critical bleeding, fibrinogen concentration in the blood becomes significantly low. If fibrinogen concentration in a patient's blood is measured to be less than 150 mg/dl, then, fresh frozen plasma or a concentrated fibrinogen preparation is administered to the patient for life support. Further, after fresh frozen plasma or a concentrated fibrinogen preparation is administered, it is necessary to examine as to whether or not fibrinogen concentration in the blood has returned to the normal range. When fibrinogen concentration in the blood has not reached the normal range after the treatment, further treatment becomes necessary for patient's life support and, therefore, this measurement needs to be performed promptly.

In the perioperative period and perinatal period, fibrinogen determination is used for the purposes as described above and, therefore, a system that can measure fibrinogen concentration in the blood with higher promptness and accuracy has been desired.

In the fibrinogen determination method involving the use of a thrombin reagent solution, the thrombin time method developed by Clauss VA is generally employed (Clauss VA, Gerinnungsphysiologische schnellmethode zur bestimmung des fibrinogens, ActaHaematologica, 17, 237-246, 1957). The thrombin time method utilizes the characteristic that the rate for fibrinogen conversion into fibrin under excessive amount of thrombin predominantly depends on the fibrinogen concentration.

This determination method comprises diluting blood plasma in a buffer, preheating the diluted solution, adding a thrombin-containing reagent solution, measuring the clotting time, and converting the obtained clotting time into fibrinogen concentration using a calibration curve prepared in advance. The clotting time according to this determination method is the period from the addition of the thrombin reagent solution to the end point. The end point is detected via an optical measurement that detects an increase in turbidity or via a physical measurement that detects an increase in viscosity.

The determination method described above and thrombin reagents used for the determination method are extensively accepted in the world and employed in clinical laboratories. However, such determination method was not necessarily suitable for use in the perioperative period and perinatal period in the following respect. For example, it is necessary to reconstitute a lyophilized thrombin reagent with purified water or the like every time for each measurement (a reconstituted solution cannot be stored over a long period of time), whole blood must be centrifuged to separate plasma therefrom, plasma must be diluted with a diluent, and the diluted plasma solution must be preheated. That is, such determination method may not necessarily have been ideal because it takes a long time before the measurement and involves a large number of steps.

An example of an improved version of the fibrinogen determination method described above is a fibrinogen determination method using a thrombin-containing dry reagent. Such method is disclosed in JP H06-094725 A (JP Patent No. 2776488) and JP H06-141895 A (JP Patent No. 2980468). A thrombin-containing dry reagent used for such determination method is prepared by adding magnetic particles to a thrombin reagent solution, dispensing a given amount of the mixture onto a reaction slide, and then lyophilizing the mixture.

The determination method involving the use of such dry reagent is characterized in that, after the addition of the sample to the reagent, a combination of an oscillating magnetic field and a static permanent magnetic field is applied at a given period (given interval), magnetic particles contained in the dry reagent are allowed to move (physically), the movement signal of the magnetic particles are detected as the amount of change in the scattered light, and the end point is detected based on the amount of change with the elapse of time (i.e., as time elapses). The period from the addition of the reagent to the end point is designated as the clotting time, and the obtained clotting time is converted to the fibrinogen concentration with a calibration curve prepared in advance.

An example of an analyzer that can implement such method is CG02N (product name; commercialized by A&T Corporation). With the use of such analyzer, a combination of an oscillating magnetic field and a static permanent magnetic field is applied at an interval (a period) of 0.5 seconds with the elapse of time, and the movement signal of the magnetic particles are monitored at the same interval (the same period).

When the analyzer described above is used, the change in the movement signal of the magnetic particles as time elapses inversely corresponds (is inversely correlated) to the change in the viscosity of the dry reagent. The end point is detected as the point at which the movement signal of the magnetic particles is attenuated by 30% from the peak value of the movement signal of the magnetic particles. Without wishing to be bound by any particular theory, the peak value of the movement signal of the magnetic particles obtained immediately after addition of the sample is considered to be the point at which the constituents of the dry reagent are completely dissolved, i.e., the point at which the viscosity in the dry reagent becomes the lowest. Let the peak value of the movement signal be designated as X and the value of the movement signal after a given period of time thereafter be designated as Y. Then, the increase in the viscosity at the time point when the attenuation in signal intensity is (X−Y)−100/X (%) is considered to be equivalent to the point at which the viscosity is X/Y times the minimal viscosity. That is, the point at which the value of the movement signal of the magnetic particles is attenuated by 30% from the peak value of the movement signal is considered to be equivalent to the point at which the viscosity is increased to 1.43 times the minimal viscosity after the addition of the sample.

In JP H06-141895 A (JP Patent No. 2980468), the technique described above is described as a fibrinogen determination method comprising mixing a fibrinogen measurement dry reagent containing a protein having thrombin activity and magnetic particles with a sample, and measuring the clotting time to determine fibrinogen concentration in the sample. In such determination method, the point at which the viscosity in the dry reagent is increased to from 1.05 times to 2.00 times the minimal viscosity is designated as the end point, and the period from the addition of the sample to the end point is designated as the clotting time.

This method is advantageous in that it is not necessary to reconstitute a lyophilized thrombin reagent with purified water or the like every time for each measurement, nor is it necessary to preheat the diluted sample. However, according to this determination method, it was necessary to dilute plasma and whole blood samples with a dedicated diluent. Thus, such determination method may not necessarily have been optimal in some aspects as a method to be employed in the perioperative period and perinatal period.

When fibrinogen concentration is determined with the thrombin-containing dry reagent disclosed in JP H06-094725 A (JP Patent No. 2776488), the clotting time obtained upon measuring undiluted plasma or undiluted whole blood is shortened to an extreme extent, and it is not possible to detect the clotting time corresponding to the fibrinogen concentration in the blood. In order to detect the clotting time corresponding to the fibrinogen concentration in the blood, accordingly, it was necessary to prolong (extend) the clotting time.

While a large number of documents including academic articles, patent applications, and manufacturers' instructions are cited herein, such disclosures are not to be deemed as being related to the patentability of the present invention.

### PRIOR ART LITERATURES

### Patent Literatures

Patent Literature 1: JP H06-094725 A (JP Patent No. 2776488)
Patent Literature 2: JP H06-141895 A (JP Patent No. 2980468)
Patent Literature 3: JP H05-219993 A (JP Patent No. 3469909)

### Non-Patent Literatures

Non-Patent Literature 1: Clauss VA: Gerinnungsphysiologische schnellmethode zur bestimmung des fibrinogens, Acta Haematologica, 17, 237-246, 1957

### SUMMARY OF THE INVENTION

In order to solve the problems as described above, the present inventors developed a novel fibrinogen measurement dry reagent and a novel method of fibrinogen determination using such dry reagent (PCT/JP2019/47592). According to this method, a sample is added to the novel fibrinogen measurement dry reagent, the detected movement signal of the magnetic particles is analyzed to determine the starting point (i.e., the starting point of the coagulation reaction), and fibrinogen concentration is then determined (quantified) using the same.

According to the method described in PCT/JP2019/47592, fibrinogen concentration in whole blood can be computed directly. However, in order to convert the fibrinogen concentration in whole blood into the fibrinogen concentration in plasma, it was necessary to perform hematocrit correction (Ht correction). Hematocrit correction is an operation performed to convert the fibrinogen concentration in whole blood into the fibrinogen concentration in plasma in accordance with the formula below.

Fibrinogen concentration in plasma=fibrinogen concentration in whole blood×(100/(100−Ht value) [Formula 1]

As represented by the formula above, the hematocrit value (Ht value) must be known in order to be able to compute the fibrinogen concentration in plasma based on the fibrinogen concentration in whole blood. However, according to the method disclosed in Patent Literature 4, however, the hematocrit value is not computed directly, and it is necessary to determine the hematocrit value using another reagent and another apparatus. This means that it is necessary to use a hematocrit measurement reagent in addition to the fibrinogen measurement reagent disclosed in Patent Literature 4. Further, an apparatus for hematocrit measurement specialized for such other hematocrit measurement reagent is necessary.

In order to solve at least part of the problems described above, it is an object of the present disclosure to provide a method and an apparatus that enable determining the fibrinogen concentration in plasma of a sample without the need of another hematocrit measurement reagent and/or another apparatus for hematocrit measurement when determining the fibrinogen concentration in plasma based on the movement level of the magnetic particles, which do not require a dilution procedure of the plasma sample or whole blood sample.

Concerning the problems described above, the present inventors have conducted concentrated studies as to whether or not the hematocrit value of the sample could be determined based on the waveform of the movement level of the magnetic particles, when determining the fibrinogen concentration in whole blood using magnetic particles regarding an undiluted citrated whole blood sample. As a result, the present inventors observed changes in the movement signal of the magnetic particles with the elapse of time after the addition of the whole blood sample dropwise to a card containing a measurement reagent and found a consistent correlation between the movement level of the magnetic particles at the point at which the movement level of the magnetic particles becomes the highest (i.e., the peak point of the waveform) and the hematocrit value of the whole blood sample. Without wishing to be bound by any particular theory, the viscosity of the mixture of the whole blood sample and the reagents becomes the lowest at the point at which the movement level of the magnetic particles becomes the highest. As such, the viscosity of such mixture at the point at which the movement level of the magnetic particles becomes the highest is considered to reflect the viscosity of the sample the most. Based on the correlation described above, the present inventors determined the hematocrit value of the whole blood sample based on the point at which the movement level of the magnetic particles becomes the highest, subjected the fibrinogen concentration in whole blood to hematocrit correction using the determined hematocrit value, computed the fibrinogen concentration in plasma of the sample, and thereby completed the present invention encompassing the same as one embodiment. In addition, the present inventors compared the determined fibrinogen concentration in plasma with the results obtained with a conventional method for determining hematocrit value and the results obtained with a conventional method for determining the fibrinogen concentration in plasma, and confirmed that the correlation is satisfactory, thereby verifying the effectiveness of the present invention.

The present disclosure encompasses the embodiments described below.

[1] A method for computing the fibrinogen concentration in plasma comprising:

(i) a step of adding a sample to a fibrinogen measurement dry reagent containing magnetic particles;

(ii) a step of allowing the magnetic particles in the reagent to move after the addition of the sample and monitoring the movement signal of the magnetic particles; and

(iii) a step of calculating a plurality of ratios of the movement signals of the magnetic particles monitored in step (ii) at a given time interval.

designating an arbitrary point within an interval during which the ratio of the movement signals of the magnetic particles calculated at a given time interval is maintained within a given range for a given period of time as the starting point, designating a point at or after the starting point at which the movement signal of the magnetic particles is attenuated by 5% to 50% from the peak value of the movement signal of the magnetic particles as the end point, designating the time from the starting point to the end point as the clotting time, and computing the fibrinogen concentration in whole blood based on the clotting time, and

computing a waveform-based hematocrit value based on the peak value of the movement signal of the magnetic particles, subjecting the computed fibrinogen concentration in whole blood to hematocrit correction using the waveform-based hematocrit value, and computing the fibrinogen concentration in plasma of the sample.

[2] The method of Embodiment 1, wherein the time interval used to calculate the ratio of the movement signals of the magnetic particles is a given time interval selected from between 0.1 seconds and 2 seconds.
[3] The method of Embodiment 1, wherein the given range of the ratio of the movement signals of the magnetic particles is 1.0±0.2.
[4] The method of any of Embodiments 1 to 3, wherein the time (time period) during which the ratio of the movement signals of the magnetic particles is maintained within a given range is 1.5 seconds.
[5] The method of any of Embodiments 1 to 4, wherein a point at or after the starting point at which the movement signal of the magnetic particles is attenuated by 20% to 30% from the peak value of the movement signal of the magnetic particles is designated as the end point.
[6] The method of any of Embodiments 1 to 5 comprising using a fibrinogen measurement dry reagent comprising:

(i) thrombin or a protein having thrombin activity;

(ii) magnetic particles;

(iii) a fibrin monomer polymerization inhibitor;

(iv) a calcium salt;

(v) a dry reagent layer solubility improving agent:

(vi) a dry reagent layer reinforcing material; and

(vii) a pH buffer.

[7] A program for executing the method of any of Embodiments 1 to 6.
[8] An information recording medium comprising the program of Embodiment 7 recorded thereon.
[9] An apparatus for fibrinogen determination comprising the program of Embodiment 7 integrated therein or the information recording medium of Embodiment 8 stored therein.

This description includes the content as disclosed in the description and/or drawings of Japanese Patent Application No. 2020-098512, which is a priority document of the present application.

### Advantageous Effects of the Invention

According to the present disclosure, the fibrinogen concentration in plasma can be computed without the need to use another hematocrit value measurement reagent and another apparatus.

### BRIEF DESCRIPTION OF THE DRAWINGS

FIG. 1 shows an example of a representative reaction slide used for a fibrinogen measurement dry reagent.

FIG. 2 is a partially exploded diagram of the reaction slide shown in FIG. 1.

FIG. 3 shows the results of the correlation test between the fibrinogen concentration in plasma and the clotting time performed in Preliminary Experiment 1. The linear relationship between the fibrinogen concentration in plasma and the clotting time is shown.

FIG. 4 shows the results of the correlation test between the results obtained by the Clauss method (the thrombin time method developed by Clauss VA: reference: Gerinnungsphysiologische schnellmethode zur bestimmung des fibrinogens, Acta Haematologica, 17, 237-246, 1957) and the results obtained with the reagent of the present disclosure performed in Preliminary Experiment 3 (the correlation with the conventional method).

FIG. 5 shows the results of the correlation test between the results of plasma measurements and the results of whole blood measurements using the reagent of the present disclosure performed in Preliminary Experiment 4 (the correlation between types of samples).

FIG. 6 shows changes in the movement signal of the magnetic particles with the elapse of time when measured using the reagent of the present disclosure (the fibrinogen measurement dry reagent of the present disclosure).

FIG. 7 shows changes in the movement signal of the magnetic particles with the elapse of time when measured using a lyophilized reagent prepared in accordance with the reagent composition according to a conventional technique.

FIG. 8 shows photographs of the appearance of dry reagent cards before and after plasma measurements.

FIG. 9 shows a calibration curve obtained by a conventional determination method performed in Preliminary Experiment 7 (Comparative Example 2).

FIG. 10 shows a calibration curve obtained by the determination method of the present disclosure performed in Preliminary Experiment 7 (the present disclosure).

FIG. 11 shows the results of the correlation test between the quantified fibrinogen value determined by the Clauss method and the quantified fibrinogen value determined by the determination method of the present disclosure performed in Preliminary Experiment 8. That is, FIG. 11 shows the correlation with the Clauss method (plasma measurements).

FIG. 12 shows the results of the correlation test between the quantified fibrinogen value determined using a citrated plasma sample and the quantified fibrinogen value determined using a citrated whole blood sample performed in Preliminary Experiment 9 (the correlation between types of samples).

FIG. 13 shows an example concerning the determination method of the present disclosure in which the period for monitoring the movement signal of the magnetic particles, the period for calculating the ratio of the movement signals of the magnetic particles, and the time interval used to calculate the ratio of the movement signals of the magnetic particles are the same.

FIG. 14 shows an example concerning the determination method of the present disclosure in which the period for monitoring the movement signal of the magnetic particles and the period for calculating the ratio of the movement signals of the magnetic particles are the same while the time interval used to calculate the ratio of the movement signals of the magnetic particles is different.

FIG. 15 shows an example concerning the determination method of the present disclosure in which the period for monitoring the movement signal of the magnetic particles, the period for calculating the ratio of the movement signals of the magnetic particles, and the time interval used to calculate the ratio of the movement signals of the magnetic particles are all different from one another.

FIG. 16 shows an example concerning the determination method of the present disclosure in which the period for monitoring the movement signal of the magnetic particles vary.

FIG. 17 shows an example concerning the determination method of the present disclosure in which the period for monitoring the movement signal of the magnetic particles and the period for calculating the ratio of the movement signals of the magnetic particles vary.

FIG. 18 shows an example concerning the determination method of the present disclosure in which the ratio of the movement signals of the magnetic particles is continuously calculated and then intermittently calculated.

FIG. 19 shows an example concerning the determination method of the present disclosure in which the ratio of the movement signals of the magnetic particles is intermittently calculated and then continuously calculated.

FIG. 20 shows a chart demonstrating changes in the movement level of the magnetic particles with the elapse of time. In FIG. 20, the first point within an interval during which the change of waveform is maintained within a given range is designated as the starting point of clotting (the hollow circle). The point at which the movement level of the magnetic particles becomes the highest is the peak point of the waveform (the hollow triangle). In FIG. 20, the end point of clotting is the point at which the movement level is attenuated by 30% from the peak point of the waveform (the hollow square). The clotting time is the time from the starting point of clotting to the end point of clotting.

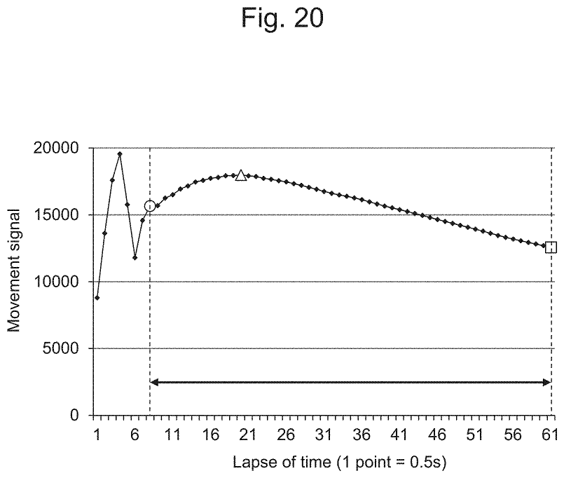

FIG. 21 shows a flow chart demonstrating the difference between the method for computing the fibrinogen concentration in plasma according to the present disclosure and the conventional method for determining the fibrinogen concentration in plasma. In FIG. 21, the measured Ht value indicates a hematocrit value measured using another measurement reagent. Further, the “waveform Ht value” indicates a hematocrit value computed based on the peak value of the waveform.

FIG. 22 shows a chart demonstrating the correlation between the measured Ht value and the peak value of the waveform.

FIG. 23 shows a chart demonstrating the correlation between the measured Ht value and the waveform Ht value.

FIG. 24 shows a chart demonstrating the correlation between the fibrinogen concentration in plasma measured with a conventional method (conventional method) and the fibrinogen concentration in plasma computed in accordance with the method of the present disclosure (novel method).

### EMBODIMENTS OF THE INVENTION

Hereafter, the present disclosure is described with reference to the drawings.

In one embodiment, the present disclosure provides a fibrinogen determination method that can be performed in the perinatal period and perioperative period. According to this method, not only can the fibrinogen concentration in whole blood of the sample be determined based on the movement signal of the magnetic particles but also, the hematocrit value can be computed based on the waveform of the movement signal of the magnetic particles without using another reagent for hematocrit value measurement or apparatus for hematocrit value measurement, the fibrinogen concentration in whole blood can be subjected to hematocrit correction using the computed hematocrit value, and the fibrinogen concentration in plasma can be computed.

### Method for Determining Fibrinogen Concentration in Whole Blood

First, the method for determining the fibrinogen concentration in whole blood is described. The method for determining the fibrinogen concentration in whole blood comprises: (i) a step of adding a sample to a fibrinogen measurement dry reagent containing magnetic particles; (ii) a step of allowing the magnetic particles in the reagent to move (physically) after the addition of the sample and monitoring the movement signal of the magnetic particles; and (iii) a step of calculating the ratio of the movement signals of the magnetic particles monitored in step (ii) at a given time interval. A plurality of ratios of the movement signals of the magnetic particles at a given time interval can be computed. An arbitrary point within an interval during which the ratio of the movement signals of the magnetic particles calculated at a given time interval is maintained within a given range for a given period of time can be designated as the starting point; a point at or after the starting point at which the movement signal of the magnetic particles is attenuated by 5% to 50% from the peak value of the movement signal of the magnetic particles can be designated as the end point; and the time (period) from the starting point to the end point can be designated as the clotting time. Step (ii) may be performed simultaneously with step (iii).

The phrase “the movement signal of the magnetic particles” used herein refers to the amount of change in the intensity of scattered light determined by, after the addition of the sample, applying a combination of an oscillating magnetic field and a static permanent magnetic field at a given interval (a given period), allowing the magnetic particles contained in the reagent to move, and applying light thereto in step (ii) (the same may be referred to as “Sₙ” herein). For convenience of description, the movement signal of the magnetic particles detected at the time point when the sample is added is designated as “S₀” herein.

The phrase “the time point for monitoring the movement signal of the magnetic particles” used herein refers to the time point at which the movement signal of the magnetic particles is measured (the same may be referred to as “mmₙ” herein). In the figures, the time point for monitoring the movement signal of the magnetic particles may be indicated by a solid circle. For the convenience of description, the time point for sample addition is designated as 0 second (mm₀) to define the time point for monitoring the movement signal of the magnetic particles herein. It is merely designated to define the time point, and the time point for sample addition may appropriately be set up, for example, −5 seconds, provided that the clotting time is computed by the method of the present disclosure. The movement signal of the magnetic particles may be monitored continuously or intermittently.

The phrase “period for monitoring the movement signal of the magnetic particles” used herein refers to the time interval of the monitoring of the movement signal of the magnetic particles. When the time points for monitoring the movement signal of the magnetic particles S₀, S₁, S₂, S₃, S₄ . . . are designated mm₀, mm₁, mm₂, mm₃, mm₄ . . . , for example, then, the period for monitoring the movement signal of the magnetic particles can be indicated as (mm₁−mm₀), (mm₂−mm₁), (mm₃−mm₂), (mm₄−mm₃) . . . . The period for monitoring the movement signal of the magnetic particles may be constant. The period for monitoring the movement signal of the magnetic particles may be altered. In the figures, the period for monitoring the movement signal of the magnetic particles may be indicated by arrows (←→) (solid thin arrows). For example, the period for monitoring the movement signal of the magnetic particles can be selected from between 0.1 seconds and 2 seconds.

In step (iii) described above, with regard to the movement signal of the magnetic particles monitored in step (ii), the ratio of the movement signals of the magnetic particles at a given time interval can be calculated. The phrase “time point for calculating the ratio of the movement signals of the magnetic particles” used herein refers to the time point at which the ratio of the movement signals of the magnetic particles is calculated (the same may be referred to as “mrₙ” herein). In the figures, the time point for calculating the ratio of the movement signals of the magnetic particles may be indicated by an outlined circle. With regard to the measurement apparatus, for example, the movement signal of the magnetic particles is measured at the time point when the sample is added (S₀), the second movement signal of the magnetic particles is measured (S₁), and, from this time point the ratio of the movement signals of the magnetic particles (S₁/S₀) can then be calculated. Such time point at which the ratio of the movement signals of the magnetic particles becomes calculable is referred to herein as “the time point for calculating the ratio of the movement signals of the magnetic particles”. In practice, because calculation is performed by an apparatus, there is a slight time lag and the time point at which S₁ was measured is different from (not strictly identical to) the time point for calculating the ratio of the movement signals of the magnetic particles mr₁. However, for the convenience of description, the time point at which the ratio of the movement signals of the magnetic particles becomes calculable is designated herein as the time point for calculating the ratio of the movement signals of the magnetic particles. Incidentally, this does not mean the apparatus must immediately calculate the ratio of the movement signals of the magnetic particles at the time point when S₁ is measured; i.e., at the time point when the ratio of the movement signals of the magnetic particles becomes calculable. For example, after S₀ and S₁ are measured; i.e., after the ratio of the movement signals of the magnetic particles becomes calculable, the apparatus may temporarily store the measured signals in a memory and then, after a given period of time, calculate the ratio of the movement signals of the magnetic particles.

The phrase “period for calculating the ratio of the movement signals of the magnetic particles” used herein refers to the period during which the ratio of the movement signals of the magnetic particles is calculated. That is, the term refers to the time interval (period) between the time point for calculating the first ratio of the movement signals of the magnetic particles and the time point for calculating the second ratio of the movement signals of the magnetic particles. Provided that the time point for monitoring the movement signal of the magnetic particles is mm₀, mm₁, mm₂, mm₃, mm₄ . . . , the time point for calculating the ratio of the movement signals of the magnetic particles is mr₁, mr₂, mr₃, mr₄ . . . , and mm₁=mr₁, mm₂=mr₂, mm₃=mr₃, mm₄=mr₄ . . . , for example, the period for calculating the ratio of the movement signals of the magnetic particles can be denoted as (mr₂−mr₁), (mr₃−mr₂), (mr₄−mr₃) . . . . In the figures, the period for calculating the ratio of the movements signal of the magnetic particles may be indicated by an outlined thick arrow. The period for calculating the ratio of the movement signals of the magnetic particles may be constant. The period for calculating the ratio of the movement signals of the magnetic particles may be altered. The period for calculating the ratio of the movement signals of the magnetic particles can be selected from 0.1 seconds to 2 seconds.

The period for monitoring the movement signal of the magnetic particles may be the same with or different from the period for calculating the ratio of the movement signals of the magnetic particles. For example, the period for monitoring the movement signal of the magnetic particles and the period for calculating the ratio of the movement signals of the magnetic particles can both be 0.5 seconds, although the periods are not limited thereto. For example, the period for monitoring the movement signal of the magnetic particles may be set as 0.1 seconds, and the period for calculating the ratio of the movement signals of the magnetic particles may be set as 0.5 seconds, although the periods are not limited thereto.

In the present description, the time interval used to calculate the ratio of the movement signals of the magnetic particles, when a signal ratio (S₂/S₁) is to be calculated, is the time interval from the time point at which S₁ is monitored to the time point at which S₂ is monitored. For example, let the time point for monitoring the movement signal of the magnetic particles be mm₀, mm₁, mm₂, mm₃, mm₄ . . . , the movement signal of the magnetic particles to be monitored at mm₀ be S₀, the movement signal of the magnetic particles to be monitored at mm₁ be S₁, the movement signal of the magnetic particles to be monitored at mm₁ be S₂, the movement signal of the magnetic particles to be monitored at mm₃ be S₃, the movement signal of the magnetic particles to be monitored at mm₄ be S₄ . . . , the time point for calculating the ratio of the movement signals of the magnetic particles be mr₁, mr₂, mr₃, mr₄ . . . , mm₁=mr₁, mm₂=mr₂, mm₃=mr₃, mm₄=mr₄, the signal ratio calculated at mr₁ be S₁/S₀, the signal ratio calculated at mr₂ be S₂/S₁, the signal ratio calculated at mr₃ be S₃/S₂, and the signal ratio calculated at mr₄ be S₄/S₃. Then, the time interval used to calculate the ratio of the movement signals of the magnetic particles would be (mm₁−mm₀), (mm₂−mm₁), (mm₃−mm₂), (mm₄−mm₃) . . . . In the figures, the time interval used to calculate the ratio of the movement signals of the magnetic particles may be indicated by a solid thick arrow. Incidentally, another signal not necessarily used for calculating the signal ratio (S₁/S₀) may be present between S₀ and S₁. In other words, it is not necessary to use all the measurement points (i.e., measured signals) for calculating the signal ratio. A constant time interval is preferably employed for calculating the ratio of the movement signals of the magnetic particles. That is, with reference to the example above, such time interval can be indicated as (mm₁−mm₀)=(mm₂−mm₁)=(mm₃−mm₂)=(mm₄−mm₃) . . . . The time interval used to calculate the ratio of the movement signals of the magnetic particles may be a constant time interval selected from between 0.1 seconds and 2 seconds, such as a time interval of 0.5 seconds, 1 second, 1.5 seconds, or 2 seconds, and may preferably be a time interval of 1 second.

An example of a reagent for fibrinogen determination to be used is a fibrinogen measurement dry reagent containing highly active thrombin or a highly active thrombin-like protein, magnetic particles, a heparin neutralizer, a fibrin monomer polymerization inhibitor, a calcium salt, an amino acid or salt thereof, or a saccharide.

As an exemplary method for preparing the fibrinogen measurement dry reagent, a buffer containing a fibrin monomer polymerization inhibitor, an amino acid or salt thereof, or a saccharide may be first prepared, highly active thrombin or a highly active thrombin-like protein may be dissolved in the buffer, magnetic particles may be added to the solution to prepare a final solution, a given amount of the final solution may be dispensed onto a reaction slide, and the solution may be frozen and lyophilized. The buffer may further contain a heparin neutralizer and/or a defoaming agent.

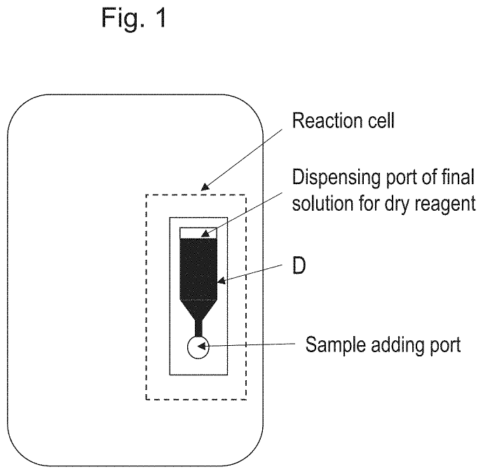

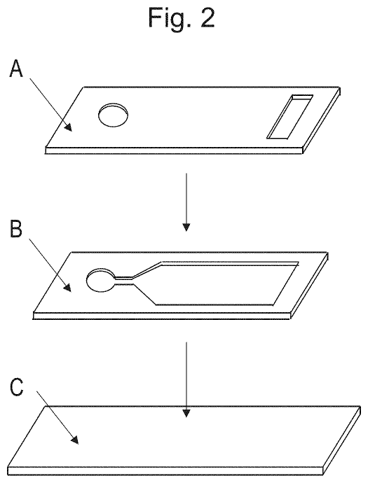

The reaction slide used in the method for preparation is not particularly limited, provided that, with the reaction slide, an increase in viscosity in the fibrinogen measurement dry reagent during fibrinogen measurement can be optically monitored as an attenuation in the movement signal of the magnetic particles. Examples include a reaction slide shown in FIGS. 1 and 2. FIG. 1 shows a top view of the reaction slide. In FIG. 1, an area surrounded by a dotted line is a reaction cell composed of a dispensing port for the final solution for preparing the fibrinogen measurement dry reagent and a sample adding port. FIG. 2 shows the structure of a reaction cell in detail. The reaction cell is constructed by first applying a transparent polyester plate B to a white polyester plate C and then applying a transparent polyester plate A to the transparent polyester plate B. First, a surfactant solution is introduced into the reaction cell through the dispensing port shown in FIG. 1 and then suction-removed to hydrophilize the region D. Then, the final solution for the fibrinogen measurement dry reagent is injected into the reaction cell through the dispensing port to fill the region D with the final solution. When this type of reaction slide is used, in general, 20 to 30 μl of the final solution for the fibrinogen measurement dry reagent can be dispensed onto the same. For methods for fibrinogen determination using magnetic particles such as this, see, for example. Patent Literature 2. The entire contents disclosed therein are incorporated herein by reference.

The reaction slide as shown in FIG. 1 may be referred to as a “dry reagent card” herein. Specifically, the fibrinogen measurement dry reagent can be applied to a dry reagent card.

Without limitation, the dry reagent layer of the fibrinogen measurement dry reagent (i) may be dissolved immediately after the sample is added dropwise thereto. Without limitation, the dry reagent layer of the fibrinogen measurement dry reagent (ii) may show no differences or substantially no difference in the dissolving rate among reagents. Without limitation, the dry reagent layer of the fibrinogen measurement dry reagent (iii) may be impact resistant (has impact resistance). Without limitation, the dry reagent layer of the fibrinogen measurement dry reagent (iv) may be uniform. Without limitation, with regard to the fibrinogen measurement dry reagent, (v) the substance that is added to satisfy the conditions (i) to (iv) above may be a substance that does not impose any influence or substantially does not impose any influence on the reaction. Without limitation, the fibrinogen measurement dry reagent may satisfy all of the conditions (i) to (v) above.

Unless indicated otherwise, contents of components of the fibrinogen measurement dry reagent described below indicate the weight and activity per 1 ml of the final solution to be dispensed onto the reaction slide shown in FIGS. 1 and 2.

Without limitation, the fibrinogen measurement dry reagent may comprise:

(i) thrombin or a protein having thrombin activity;

(ii) magnetic particles:

(iii) a fibrin monomer polymerization inhibitor;

(iv) a calcium salt;

(v) a dry reagent layer solubility improving agent:

(vi) a dry reagent layer reinforcing material; and

(vii) a pH adjuster (pH buffer),

as essential components. Without limitation, the fibrinogen measurement dry reagent may further comprise, as optional components, a heparin neutralizer and/or a defoaming agent. Without limitation, the fibrinogen measurement dry reagent may be for use in measuring an undiluted plasma or whole blood sample.

The phrase “undiluted whole blood” used herein refers to whole blood, which is not subjected to any dilution procedure, such as the addition of a dilution buffer, to the whole blood sample after blood sampling. As such, even if the blood is diluted with a citrate solution or other substances contained in the blood collection tube at the time of blood sampling (such blood is generally referred to as “citrated whole blood”) so long as the whole blood sample is not subjected to any specific dilution procedure after blood sampling, such blood is within the scope of “undiluted whole blood” as used herein. As such, undiluted whole blood encompasses citrated whole blood and heparinized whole blood that are not subjected to any dilution procedure. The phrase “undiluted plasma” used herein refers to a supernatant obtained by centrifugation of undiluted whole blood, and such plasma is not subjected to a dilution procedure, such as the addition of a dilution buffer. As such, the undiluted plasma encompasses citrated plasma and heparinized plasma that are not subjected to a dilution procedure. Incidentally, the phrase “non-diluted” is synonymous with the term “undiluted” herein.

Without limitation, the fibrinogen measurement dry reagent may comprise thrombin or a protein having thrombin activity. A protein having thrombin activity may be referred to as a “thrombin-like protein” herein. The phrase “thrombin activity” used herein refers to activity capable of catalyzing both the reactions: (i) conversion of fibrinogen into a fibrin monomer; and (ii) activation of factor XIII into factor XIIIa in the presence of a calcium ion. A protein having such activity is referred to as a protein having thrombin activity. It should be noted, however, that a single protein need not necessarily enhance both the reactions (i) and (ii) above. In other words, a mixture of (i) a first protein having thrombin activity of catalyzing the conversion of fibrinogen into fibrin monomer(s) and (ii) a second protein having thrombin activity of catalyzing the activation of factor XIII into factor XIIIa can be used. An example of the first protein is snake venom thrombin-like enzymes. The second protein may be a protein having activity of specifically cleaving a site between arginine 37 and glycine 38 from the N terminus of the factor XIII A subunit. Examples of thrombin or a protein having thrombin activity include, but are not limited to, bovine thrombin, human thrombin, and recombinants thereof. In some embodiments, thrombin or a protein having thrombin activity may be bovine thrombin. Bovine thrombin that is widely commercialized and readily available in the form of a lyophilized product may be used. Examples of thrombin or a protein having thrombin activity include, but are not limited to, a combination of snake venom thrombin-like enzymes and a protein having activity of specifically cleaving a site between arginine 37 and glycine 38 from the N terminus of the factor XIII A subunit. While the activity of thrombin or a protein having thrombin activity to be incorporated into the fibrinogen measurement dry reagent is not particularly limited, for example, the bovine thrombin activity level may be selected from the range of 100 to 500 NIHU/ml of the final solution, with the range of 150 to 400 NIHU/ml of the final solution being preferable.

Without limitation, the fibrinogen measurement dry reagent may comprise magnetic particles. Any conventional magnetic particles may be used for the fibrinogen measurement dry reagent without limitation. Examples of magnetic particles include, but are not limited to, triiron tetraoxide particles, iron sesquioxide particles, iron particles, cobalt particles, nickel particles, and chromium oxide particles. For example, magnetic particles can be fine particles of triiron tetraoxide. For example, fine particles of triiron tetraoxide are preferably used from the perspective of the intensity of the movement signal of the magnetic particles. The particle diameter of the magnetic particles is not particularly limited, and the average particle diameter can be 0.05 to 5 μm, 0.1 to 3.0 μm, such as 0.25 to 0.5 μm, although the particle diameter is not limited thereto. Without limitation, the average particle diameter of the magnetic particles may be 0.1 to 3.0 μm. The phrase “average particle diameter” used herein refers to a particle diameter (D50) at a cumulative value of 50% in a particle size distribution by a laser diffraction scattering method, unless otherwise specified. The magnetic particle content in the fibrinogen measurement dry reagent is not particularly limited. For example, such content may preferably be 4 to 40 mg/ml of the final solution.

Without limitation, the fibrinogen measurement dry reagent may comprise, as an optional component, a heparin neutralizer. Any conventional heparin neutralizer may be used without limitation, and examples thereof include, but are not limited to, polybrene, protamine sulfate, and heparinase. For example, polybrene may preferably be used as the heparin neutralizer from the perspective of good storage stability and cost effectiveness. The amount of a heparin neutralizer to be incorporated into a fibrinogen measurement dry reagent is not particularly limited and may be appropriately determined. When polybrene is used as a heparin neutralizer, for example, the amount of polybrene to be incorporated into the fibrinogen measurement dry reagent may preferably be 50 to 300 μg/ml of the final solution.

Without limitation, the fibrinogen measurement dry reagent may comprise a fibrin monomer polymerization inhibitor. Any conventional fibrin monomer polymerization inhibitor may be used (contained) in the fibrinogen measurement dry reagent without particular limitation. Examples of fibrin monomer polymerization inhibitors include, but are not limited to, GPRP (glycine-proline-arginine-proline) peptide and derivatives thereof, such as GPRP-amide, and GHRP (glycine-histidine-arginine-proline) peptide and derivatives thereof, such as GHRP-amide. In other embodiments, the fibrin monomer polymerization inhibitor can be GPRPA (glycine-proline-arginine-proline-alanine) peptide and derivatives thereof, such as GPRPA-amide. Without limitation, the fibrin monomer polymerization inhibitor may preferably be GPRP peptide and derivatives thereof from the perspective of affinity to fibrinogen. Such peptide is an analog of knob ‘A’ which is exposed when thrombin reacts with fibrinogen and fibrinopeptide A becomes released from the a chain of fibrinogen. When such peptide binds to hole ‘a’ that is present in the y chain instead of knob ‘A,’ the same inhibits fibrin monomer polymerization (John WW: Mechanisms of fibrin polymerization and Clinical implications, Blood, 121 (10), 1712-1719, 2013).

The amount of a fibrin monomer polymerization inhibitor to be incorporated into a fibrinogen measurement dry reagent may appropriately be determined without particular limitation. When GPRP-amide is used as the fibrin monomer polymerization inhibitor, the amount of the GPRP-amide to be incorporated into the fibrinogen measurement dry reagent may preferably be 100 to 300 μg/ml of the final solution.

Without limitation, the fibrinogen measurement dry reagent may comprise a calcium salt. Any conventional calcium salt may be used for the dry reagent without limitation. Examples of inorganic acid calcium salts include calcium chloride, calcium nitrite, calcium sulfate, and calcium carbonate. Examples of organic acid calcium salts include calcium lactate and calcium tartrate. Without limitation, calcium chloride is preferable as the calcium salt. The amount of calcium salt to be incorporated into a fibrinogen measurement dry reagent may appropriately be determined without particular limitation. When a calcium chloride dihydrate is used as the calcium salt, the amount of a calcium chloride dihydrate to be incorporated into the fibrinogen measurement dry reagent is preferably 0.2 to 2 μg/ml of the final solution.

Without limitation, the fibrinogen measurement dry reagent may comprise a dry reagent layer solubility improving agent. Examples of the dry reagent layer solubility improving agent include an amino acid or salt thereof and a saccharide. The amino acid or salt thereof or a saccharide used herein may be any of a neutral amino acid or salt thereof, an acidic amino acid or salt thereof, a basic amino acid or salt thereof, a monosaccharide, and a polysaccharide. Examples of representative acidic amino acids or salts thereof include glutamic acid, sodium glutamate, aspartic acid, and sodium aspartate. Examples of representative neutral amino acids or salts thereof include glycine, glycine hydrochloride, and alanine. Examples of representative basic amino acids or salts thereof include lysine, lysine hydrochloride, and arginine. Examples of monosaccharides include glucose and fructose. Examples of polysaccharides include sucrose, lactose, and dextrin. Among these substances, glycine is the most preferable from the perspective of good solubility of the reagent when a sample is added to the fibrinogen measurement dry reagent, good reproducibility of the movement signals of magnetic particles, and good impact resistance. Without limitation, specifically, the dry reagent layer solubility improving agent may be glycine.

The amount of the dry reagent layer solubility improving agent to be incorporated into the fibrinogen measurement dry reagent, such as the amount of an amino acid or salt thereof or a saccharide, may appropriately be determined without particular limitation. When glycine is used as the dry reagent layer solubility improving agent, the amount of glycine to be incorporated into the fibrinogen measurement dry reagent may be 1.5% by weight or more, 1.6% by weight or more, 1.7% by weight or more, 1.8% by weight or more, 1.9% by weight or more, 2.0% by weight or more, 2.1% by weight or more, 2.2% by weight or more, 2.3% by weight or more, 2.4% by weight or more, 2.5% by weight or more, 2.6% by weight or more, 2.7% by weight or more, 2.8% by weight or more, 2.9% by weight or more, 3.0% by weight or more, 3.1% by weight or more, 3.2% by weight or more, 3.3% by weight or more, 3.4% by weight or more, 3.5% by weight or more, 3.6% by weight or more, 3.7% by weight or more, 3.8% by weight or more, 3.9% by weight or more, 4.0% by weight or more, 4.1% by weight or more, 4.2% by weight or more, 4.3% by weight or more, 4.4% by weight or more, 4.5% by weight or more, 4.6% by weight or more, 4.7% by weight or more, 4.8% by weight or more, or 4.9% by weight or more, such as 5.0% by weight. When glycine is used as the dry reagent layer solubility improving agent, the amount of glycine to be incorporated into the fibrinogen measurement dry reagent may be 5.0% by weight or less, 4.9% by weight or less, 4.8% by weight or less, 4.7% by weight or less, 4.6% by weight or less, 4.5% by weight or less, 4.4% by weight or less, 4.3% by weight or less, 4.2% by weight or less, 4.1% by weight or less, 4.0% by weight or less, 3.9% by weight or less, 3.8% by weight or less, 3.7% by weight or less, 3.6% by weight or less, 3.5% by weight or less, 3.4% by weight or less, 3.3% by weight or less, 3.2% by weight or less, 3.1% by weight or less, 3.0% by weight or less, 2.9% by weight or less, 2.8% by weight or less, 2.7% by weight or less, 2.6% by weight or less, 2.5% by weight or less, 2.4% by weight or less, 2.3% by weight or less, 2.2% by weight or less, 2.1% by weight or less, 2.0% by weight or less, 1.9% by weight or less, 1.8% by weight or less, 1.7% by weight or less, or 1.6% by weight or less, such as 1.5% by weight. The amount of glycine to be incorporated into the fibrinogen measurement dry reagent herein encompasses any combination of the minimal amount and the maximal amount wherein the minimal amount and the maximal amount are set to be any of the minimal amounts and the maximal amounts mentioned above. For example, the amount of glycine to be incorporated into the fibrinogen measurement dry reagent may be set as 1.5% to 5.0% by weight, 2.0% to 5.0% by weight, 2.5% to 5.0% by weight, 3.0% to 5.0% by weight, 3.5% to 5.0% by weight, 4.0% to 5.0% by weight, 4.5% to 5.0% by weight, 1.5% to 4.5% by weight, 2.0% to 4.5% by weight, 2.5% to 4.5% by weight, 3.0% to 4.5% by weight, 3.5% to 4.5% by weight, 4.0% to 4.5% by weight, 1.5% to 4.0% by weight, 2.0% to 4.0% by weight, 2.5% to 4.0% by weight, 3.0% to 4.0% by weight, 3.5% to 4.0% by weight, 1.5% to 3.5% by weight, 2.0% to 3.5% by weight, 2.5% to 3.5% by weight, 3.0% to 3.5% by weight, 1.5% to 3.0% by weight, 2.0% to 3.0% by weight, 2.5% to 3.0% by weight, 1.5% to 2.5% by weight, 2.0% to 2.5% by weight, or 1.5% to 2.0% by weight. Without limitation, when glycine is used as the dry reagent layer solubility improving agent, the amount of glycine to be incorporated into the fibrinogen measurement dry reagent is preferably 1.5% to 4.0% by weight. Without limitation, when glycine is used as the dry reagent layer solubility improving agent, the amount of glycine to be incorporated into the fibrinogen measurement dry reagent is preferably 2.0% to 3.0% by weight. When glycine is used as the dry reagent layer solubility improving agent for measuring an undiluted plasma sample, the amount of glycine to be incorporated into the fibrinogen measurement dry reagent may be within the range mentioned above, such as 1.5% to 4.0% by weight. When glycine is used as the dry reagent layer solubility improving agent for measuring an undiluted whole blood sample, the amount of glycine to be incorporated into the fibrinogen measurement dry reagent may be within the range mentioned above, such as 1.5% by weight or more. When glycine is used as the dry reagent layer solubility improving agent for measuring an undiluted whole blood sample, for example, the amount of glycine to be incorporated into the fibrinogen measurement dry reagent may be 1.5% to 5.0% by weight, or 1.5% to 4.5% by weight, such as 1.5% to 4.0% by weight. When enabling measurement of both an undiluted plasma sample and an undiluted whole blood sample, when glycine is used as the dry reagent layer solubility improving agent, the amount of glycine to be incorporated into the fibrinogen measurement dry reagent may be any combination of these various ranges. It should be noted that the unit “% by weight” used herein indicates the concentration in the final solution; i.e., the final concentration, unless otherwise specified.

Without limitation, the fibrinogen measurement dry reagent comprises a pH buffer (may be referred to as a “pH adjuster”). Prior to lyophilization, a buffer supplemented with a protein having thrombin activity, magnetic particles, a heparin neutralizer, a fibrin monomer polymerization inhibitor, a calcium salt, and a dry reagent layer solubility improving agent is not particularly limited, provided that buffering actions at pH 6.0 to 8.0. In some embodiments, a pH adjusting agent (pH buffer) may be capable of adjusting the pH level of the reagent to a pH of 6.0 to 8.0, such as about pH 7.35 or about pH 7.5. Examples of preferable buffers include 40 mM HEPES buffer (pH=7.35) and 40 mM Tris-HCl buffer (pH=7.5).

Without limitation, the fibrinogen measurement dry reagent comprises a dry reagent layer reinforcing material (a material for reinforcing the dry reagent layer). Examples of the dry reagent layer reinforcing material include, but are not limited to, bovine serum albumin and human serum albumin. When bovine serum albumin is used as the dry reagent layer reinforcing material, the amount of the dry reagent layer reinforcing material to be incorporated into the dry reagent may preferably be in a range of 0.6 to 2.0 mg/l ml of the final solution.

Without limitation, the fibrinogen measurement dry reagent may comprise, as an optional component, a defoaming agent. Examples of the defoaming agent include, but are not limited to, sorbitan monolaurate, a silicone-based defoaming agent, and a polypropylene glycol-based defoaming agent. When sorbitan monolaurate is used as the defoaming agent, the amount of the defoaming agent to be incorporated into the dry reagent may preferably be in a range of about 0.001% to about 0.010% by weight.

The method of drying the buffer containing the components described above is preferably lyophilization, from the perspective of solubility of a fibrinogen measurement dry reagent, the movement signal intensity of magnetic particles, and reproducibility. When the buffer containing the components described above is air-dried, solubility of the reagents is poor, the movement signals of magnetic particles are weak, and, therefore, it is difficult to detect the end point. Further, when the buffer containing the components described above is air-dried, the clotting time determined based on the end point (even if detected) may not necessarily correspond to the fibrinogen concentration.

The method of freezing and lyophilization are not particularly limited. Common techniques of freezing can be employed. For example, a final solution for the fibrinogen measurement dry reagent is dispensed onto a reaction slide through the dispensing port shown in FIG. 1. Thereafter, the reaction slide is stored and frozen in a freezer maintained at −40° C. or lower for one whole day and night, the reaction slide is mounted on a lyophilizer in which the shelf temperature is set at −40° C. or lower and stored and frozen therein for one whole day and night, or the reaction slide is frozen instantly with liquid nitrogen. In addition, the technique for lyophilizing the frozen reaction slide is not particularly limited. To exemplify the lyophilizing method, the lyophilizing method includes a method in which the temperature of the frozen reaction slide may be linearly raised from −30° C. to −20° C. over a period of 24 hours in vacuum, the temperature thereof may be linearly raised from −20° C. to 30° C. over a period of 20 hours, the temperature may be maintained at 30° C. for 3 hours, and dry air may then be applied to release the vacuum.

It is preferable to immediately seal the lyophilized fibrinogen measurement dry reagent with an aluminum film in a dehumidified environment. While the dehumidified environment is not particularly limited, an environment in which temperature is at room temperature of 22° C. to 27° C. and relative humidity is 35% or lower is preferable. While specifications of the aluminum film are not particularly limited, a preferable aluminum film may be a 5-layer-structure aluminum film (thickness: 86 μm) comprising a polyester film (thickness: 12 μm), polyethylene resin (thickness: 15 μm), an aluminum foil (thickness: 9 μm), a polyethylene resin (thickness: 20 μm), and a polyethylene film (thickness: 30 μm) adhered with an AC coating agent. The entire fibrinogen measurement dry reagent is wrapped with the aluminum foil and sealed via heat adhesion. It is preferable to refrigerate the fibrinogen measurement dry reagent in a sealed state before using the same for fibrinogen measurement.

Fibrinogen determination involving the use of the fibrinogen measurement dry reagent may be performed by adding an sample to the reagent to dissolve the reagent and using an apparatus that applies a combination of an oscillating magnetic field and a static permanent magnetic field to allow the magnetic particles contained in the reagent to move, detects the movement signal of the magnetic particles as the amount of change in the scattered light, detects the clotting point based on the change with the elapse of time, and computes the clotting time as the time from the starting point (the starting point of the coagulation reaction) to the clotting point. The obtained clotting time is correlated with the fibrinogen concentration in the sample.

With regard to the fibrinogen determination method, the given range of the ratio of the movement signals of the magnetic particles is not particularly limited. For example, the given range of the ratio of the movement signals of the magnetic particles can be in a range from 1.0±0.05 to 1.0±0.2, such as 1.0±0.2, 1.0±0.19, 1.0±0.18, 1.0±0.17, 1.0±0.16, 1.0±0.15, 1.0±0.14, 1.0±0.13, 1.0±0.12, 1.0±0.11, 1.0±0.1, 1.0±0.09, 1.0±0.08, 1.0±0.07, 1.0±0.06, or 1.0±0.05. Without limitation, the given range of the ratio of the movement signals of the magnetic particles may preferably be 1.0±0.05 to 1.0±0.15 and particularly preferably 1.0±0.1 from the perspective of good reproducibility of the clotting time. In other words, the given range of the ratio of the movement signals of the magnetic particles may be, for example, a range of 0.8 to 1.2, a range of 0.81 to 1.19, a range of 0.82 to 1.18, a range of 0.83 to 1.17, a range of 0.84 to 1.16, a range of 0.85 to 1.15, a range of 0.86 to 1.14, a range of 0.87 to 1.13, a range of 0.88 to 1.12, a range of 0.89 to 1.11, a range of 0.9 to 1.1, a range of 0.91 to 1.09, a range of 0.92 to 1.08, a range of 0.93 to 1.07, a range of 0.94 to 1.06, or a range of 0.95 to 1.05. A range of 0.9 to 1.1 is particularly preferable from the perspective of good reproducibility of the clotting time.

With regard to the fibrinogen determination method, the time (the interval) during which the ratio of the movement signals of the magnetic particles is maintained within a given range is not particularly limited. For example, the time during which the ratio of the movement signals of the magnetic particles is maintained within a given range can be 1 to 5 seconds, 1 to 4 seconds, 1 to 3 seconds, 5 seconds, 4.5 seconds, 4 seconds, 3.5 seconds, 3 seconds, 2.5 seconds, 2 seconds, 1.5 seconds, or 1 second, although the time is not limited thereto. Without limitation, the time during which the ratio of the movement signals of the magnetic particles is maintained within a given range may preferably be 1 to 3 seconds, and more preferably 1.5 seconds, from the perspective of good reproducibility of the clotting time.

With regard to the fibrinogen determination method, the starting point is an arbitrary point within an interval during which a plurality of ratios of the movement signals of the magnetic particles are maintained within a given time interval and the ratio is maintained within a given range for a given period of time. The ratio of the movement signals of the magnetic particles at a given time interval can be monitored continuously or intermittently. Without limitation, the first point within the time period (interval) during which the ratio of the movement signals of the magnetic particles is maintained within a given range for a given period of time can be designated as the starting point. Without limitation, the starting point can be a point other than the first point within the time period during which the ratio is maintained within a given range for a given period of time, such as the second, the third, or the fourth point within the time period during which the rate is maintained within a given range for a given period of time. In order to avoid the initial variability of the signals after the addition of the sample, the starting point is defined in the method of the present disclosure for the convenience of description and, for example, such point is described as the point of measurement time 0 (sec) in the tables. However, this does not mean that the coagulation reaction is not initiated at all before the point of measurement time 0 (sec).

With regard to the fibrinogen determination method, unless otherwise specified, the peak value is the peak value of the movement signal of the magnetic particles observed at or after the starting point and this is the maximal movement signal of the magnetic particles among the signals of the magnetic particles at or after the starting point. This peak value is different from the peak value according to conventional techniques. That is, according to the method described in JP H06-141895 A (JP Patent No. 2980468), the maximal signal among all the measured signals was simply designated as the peak value. However, when the present inventors applied the dry reagent described in Preliminary Experiment 1 to the determination method according to JP H06-141895 A (JP Patent No. 2980468), the movement signal of the magnetic particles varied to a significant extent in the initial measurement stage after addition of the sample. When the maximal signal among all the measured signals was designated as the peak value, there were instances where fibrinogen determination could not be performed correctly. Therefore, the starting point is defined, and the peak value of the movement signal of the magnetic particles at or after the starting point is correctly defined, thereby determining fibrinogen more accurately with regard to undiluted samples.

With regard to the fibrinogen determination method, the end point is an arbitrary point among the points where the signal is attenuated by 5% to 50% from the peak value of the movement signal of the magnetic particles at or after the starting point defined in the manner described above. For example, when the peak value of the movement signal of the magnetic particles at or after the starting point is designated as 100%, a point at which the movement signal of the magnetic particles is equivalent to 70% of the peak value of the movement signal is referred to as a point attenuated by 30% herein (point attenuated by 30% from the peak value). For example, the end point can be a point attenuated by 5% to 50%, a point attenuated by 10% to 45%, a point attenuated by 15% to 40%, a point attenuated by 20% to 35%, a point attenuated by 20% to 30%, such as a point attenuated by 20%, a point attenuated by 25%, or a point attenuated by 30%, from the peak value of the movement signal of the magnetic particles at or after the starting point, although the end point is not limited thereto. A point attenuated by 30% from the peak value of the movement signal of the magnetic particles is particularly preferable from the perspective of good reproducibility of the clotting time. Without limitation, the end point can be defined depending on the type of sample; i.e., whether the blood sample to be measured is an undiluted whole blood sample or undiluted plasma sample. That is, when an undiluted whole blood sample is to be measured, the end point can be a point attenuated by 20% from the peak value of the movement signal of the magnetic particles at or after the starting point. For example, when the blood sample to be measured is an undiluted plasma sample, the end point can be a point attenuated by 30% from the peak value of the movement signal of the magnetic particles at or after the starting point. Each end point applied according to the different sample type can appropriately be selected from among the points attenuated by 5% to 50% from the peak value of the movement signal of the magnetic particles at or after the starting point. Incidentally, the phrase “the peak value of the movement signal of the magnetic particles at or after the starting point” used herein refers to the maximal signal (C) among the movement signals of the magnetic particles measured at or after the starting point, and this may include the starting point itself. In other words, if the movement signal of the magnetic particles at the starting point is the maximal signal among the movement signals of the magnetic particles measured at or after the starting point, then such movement signal is the peak value of the movement signals of the magnetic particles at or after the starting point.

The phrase “clotting time” used herein refers to the time from the starting point to the end point. That is, with regard to the fibrinogen determination method disclosed herein, the clotting time is computed as the time from the starting point to the end point. The obtained clotting time is correlated with the fibrinogen concentration. Examples of an apparatus that can implement the fibrinogen determination method disclosed herein include CG02N (product name; commercialized by A&T Corporation), although apparatuses that can be used are not limited thereto.

CG02N is an apparatus suitable for a conventional fibrinogen determination method (JP H06-141895 A (JP Patent No. 2980468)). After a sample is added to the fibrinogen measurement dry reagent, a combination of an oscillating magnetic field and a static permanent magnetic field is applied at intervals of 0.5 seconds, and the movement signals of the magnetic particles are monitored at the same intervals. In order to implement the fibrinogen determination method disclosed herein with such apparatus, in addition to the foregoing, in particular embodiments, for example, the ratio of the movement signals of the magnetic particles is continuously computed at intervals of 1 second, and the first point within the interval during which the ratio is maintained within a range of 1.0±0.1 for 1.5 seconds can be detected as the starting point. In this regard, a point attenuated by 5% to 50%, such as a point attenuated by 30%, from the peak value of the movement signal of the magnetic particles at or after the starting point may be designated as the end point, and the time from the starting point to the end point may be computed as the clotting time. It should be noted that this is merely one example and the fibrinogen measurement method is not limited thereto.

A series of operation including such arithmetic processing may be carried out by controlling the apparatus with a program or software. The program or software may be integrated in the apparatus or recorded on an information recording medium. In one embodiment, the present disclosure provides a program or software for executing (implementing) the fibrinogen determination method. In one embodiment, the present disclosure provides an information recording medium comprising the program or software recorded thereon. In one embodiment, the present disclosure provides an apparatus for fibrinogen determination comprising a program or software for executing the fibrinogen determination method integrated therein or the information recording medium stored therein. In some embodiments, the apparatus for fibrinogen determination encompasses an apparatus comprising the program of the present disclosure integrated in the CG02N apparatus.

Table 1 shows an example of whole blood sample measurements performed by the fibrinogen determination method according to the present disclosure. In such method, monitoring of the movement signal of the magnetic particles is initiated immediately after the addition of the sample and monitoring is performed at intervals of 0.5 seconds. That is, the period for monitoring the movement signal of the magnetic particles is 0.5 seconds. The ratio of the movement signals of the magnetic particles is then continuously computed at intervals of 1 second. In other words, the time interval used to compute the ratio of the movement signals of the magnetic particles is 1 second. That is, the ratio of the movement signals of the magnetic particles is computed as follows: (the movement signal of the magnetic particles detected at the monitoring time of 1.0 second)/(the movement signal of the magnetic particles detected at the monitoring time of 0 seconds), (the movement signal of the magnetic particles detected at the monitoring time of 1.5 seconds)/(the movement signal of the magnetic particles detected at the monitoring time of 0.5 seconds), (the movement signal of the magnetic particles detected at the monitoring time of 2.0 seconds)/(the movement signal of the magnetic particles detected at the monitoring time of 1.0 second) . . . . The interval during which the ratio is maintained within a range of 1.0±0.1 for 1.5 seconds is the period of the monitoring time of 5.0 to 6.5 seconds. The first point thereof is the monitoring time of 5.0 seconds, and this point can be thus designated as the starting point (the starting point of the coagulation reaction: the time point of measurement time 0 (sec)). The peak value of the movement signal of the magnetic particles at or after the starting point is 2726c detected at the monitoring time of 7.0 seconds. The movement signal of the magnetic particles that is lower by 30% than the peak value of the movement signal of the magnetic particles at or after the starting point is computed to be 1908c. That is, the end point is the point at which the movement signal of the magnetic particles is 1908c, and the clotting time is computed to be 20.1 seconds. Since the movement signal of the magnetic particles 1908c is a computed value, the corresponding monitoring time and ratios of the movement signals of the magnetic particles at intervals of 1 second are not shown in the table. That is, the clotting time determined by the method of the present disclosure is not necessarily one of the actual measurement points (one of the actual monitoring time points).

TABLE 1

| | | Ratio of movement signal of magnetic | | |
|---|---|---|---|---|
| Time point for monitoring the | Movement signal of | particles at a time interval of 1 sec | Start/ | Measurement |
| movement signals of | magnetic particles | for computing the ratio of movement | peak/ | time |
| magnetic particles (sec) | (C) | signals of magnetic particles (—) | end | (sec) |
| 0 | 1430 | — | | |
| 0.5 | 86 | — | | |
| 1.0 | 359 | 0.25 | | |
| 1.5 | 114 | 1.33 | | |
| 2.0 | 3722 | 10.37 | | |
| 2.5 | 4235 | 37.15 | | |
| 3.0 | 1841 | 0.49 | | |
| 3.5 | 3534 | 0.83 | | |
| 4.0 | 2890 | 1.57 | | |
| 4.5 | 2389 | 0.68 | | |
| 5.0 | 2673 | 0.92 | Start | 0 |
| 5.5 | 2581 | 1.08 | | 0.5 |
| 6.0 | 2682 | 1.00 | | 1.0 |
| 6.5 | 2651 | 1.03 | | 1.5 |
| 7.0 | 2726 | 1.02 | Peak | 2.0 |
| 7.5 | 2678 | 1.01 | | 2.5 |
| 8.0 | 2721 | 1.00 | | 3.0 |
| 8.5 | 2673 | 1.00 | | 3.5 |
| 9.0 | 2708 | 1.00 | | 4.0 |
| 9.5 | 2665 | 1.00 | | 4.5 |
| 10.0 | 2677 | 0.99 | | 5.5 |
| 10.5 | 2635 | 0.99 | | 6.0 |
| 11.0 | 2635 | 0.98 | | 6.5 |
| . | . | . | | . |
| . | . | . | | . |
| . | . | . | | . |
| 22.0 | 2031 | 0.98 | | 17.5 |
| 22.5 | 2007 | 0.98 | | 18.0 |
| 23.0 | 1980 | 0.97 | | 18.5 |
| 23.5 | 1957 | 0.98 | | 19.0 |
| 24.0 | 1940 | 0.98 | | 19.5 |
| 24.5 | 1913 | 0.98 | | 20.0 |
| — | 1908 | — | End | 20.1 |
| 25.0 | 1889 | 0.97 | | 20.5 |
| 25.5 | 1870 | 0.98 | | 21.0 |

Peak movement signal of magnetic particles at or after the starting point: 2726
Movement signal of magnetic particles attenuated by 30% from the peak movement signal of magnetic particles at or after the starting point: 1908
Clotting time = 20.1 sec

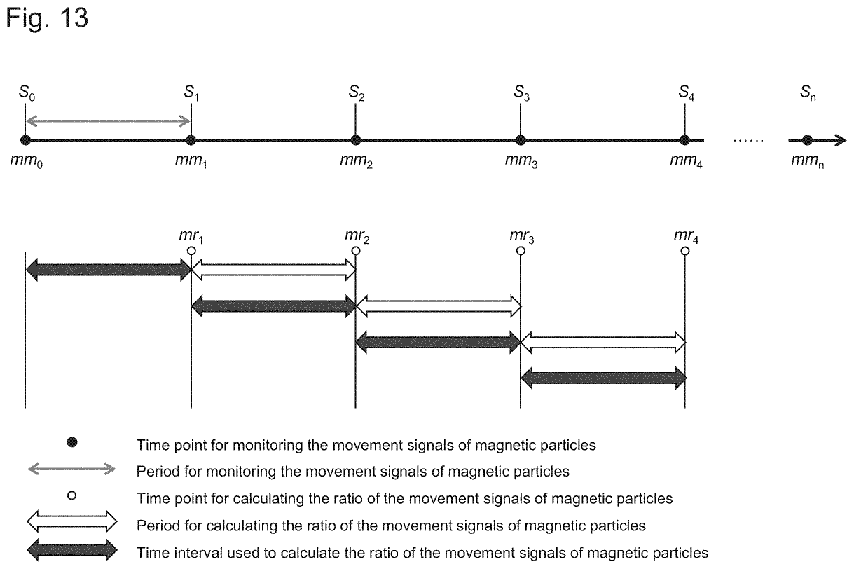

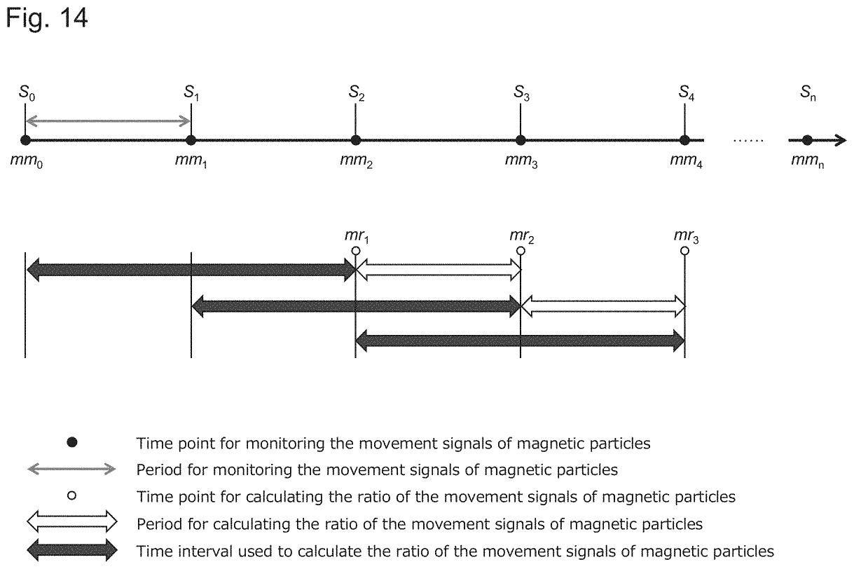

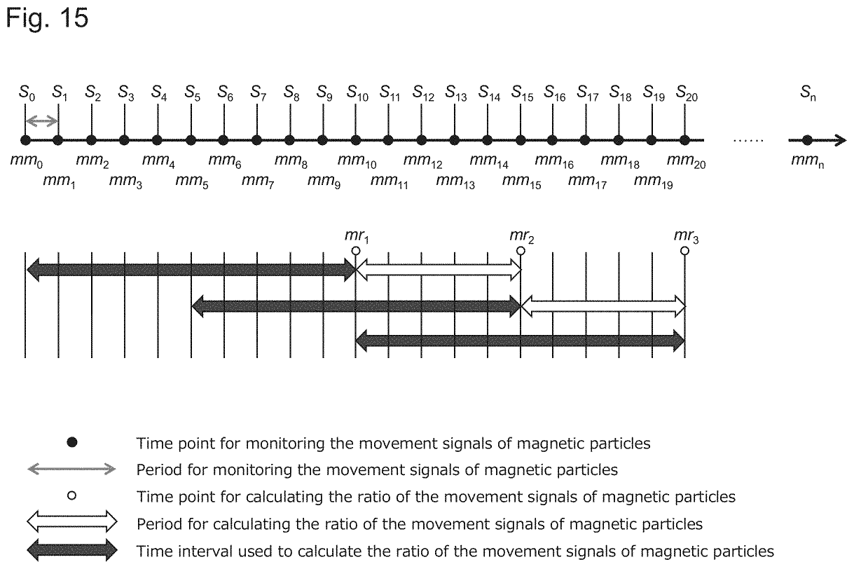

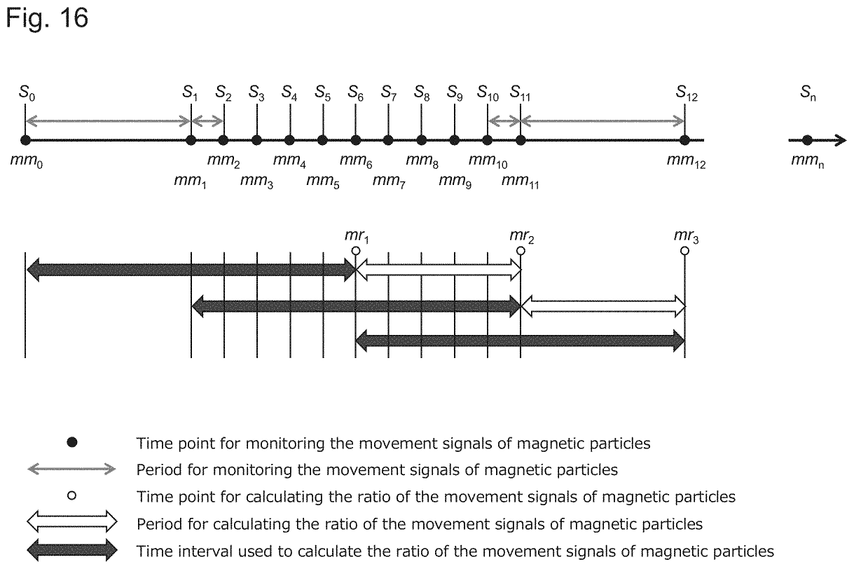

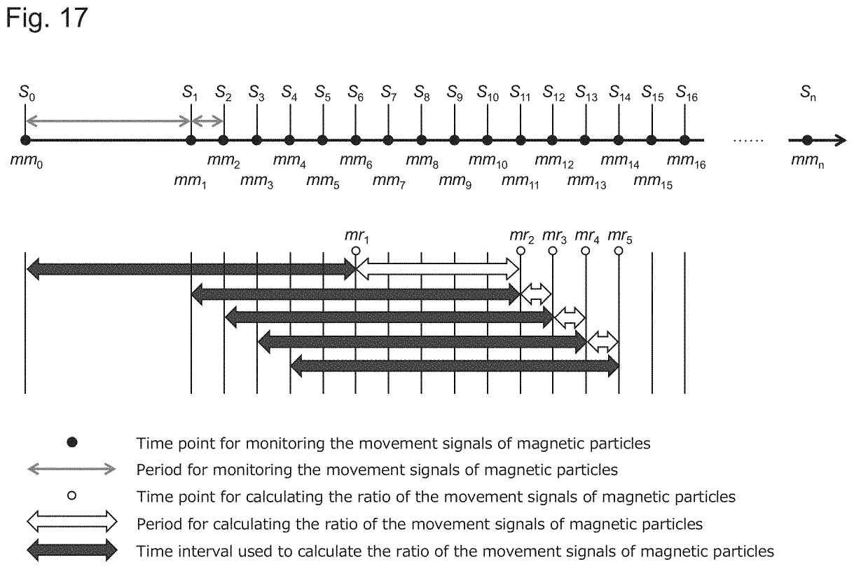

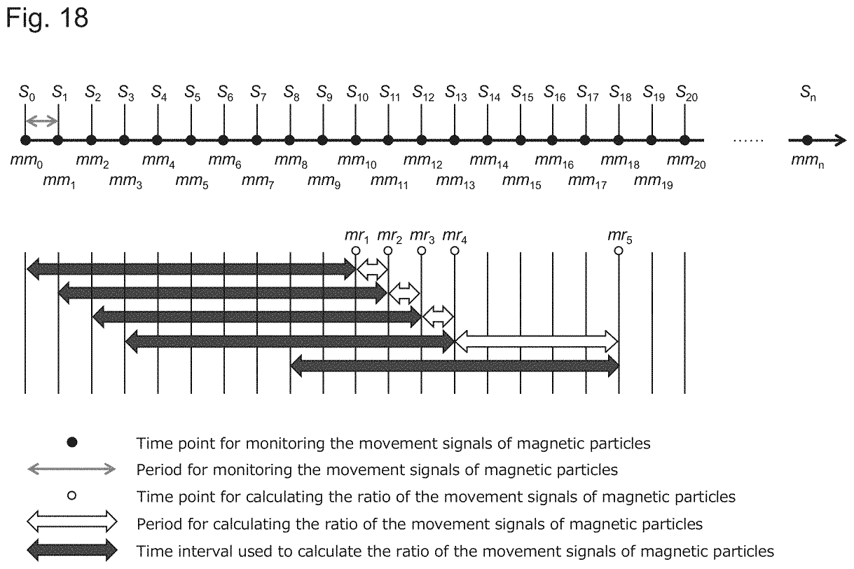

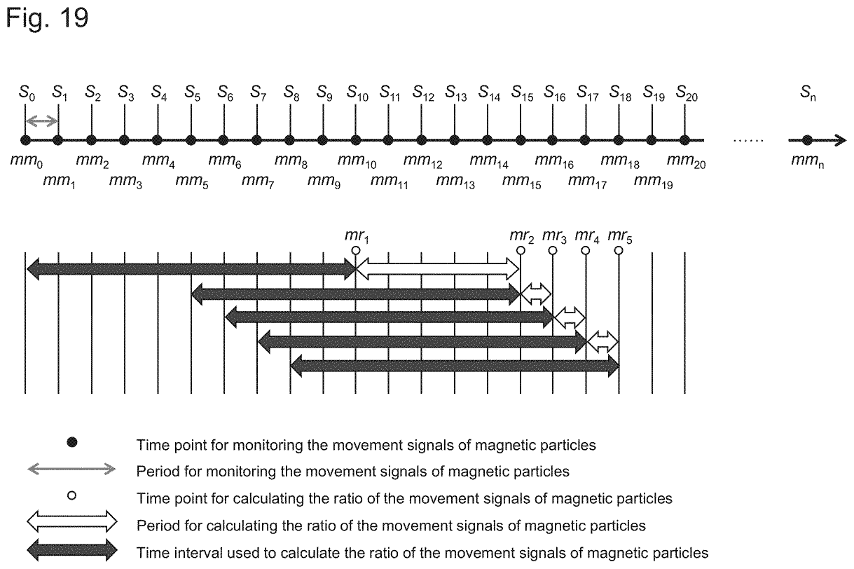

Incidentally, the fibrinogen determination method is not limited to the above. The period for monitoring the movement signal of the magnetic particles, the period for computing the ratio of the movement signals of the magnetic particles, and the time interval used to compute the signal ratio of magnetic particles may all be the same (e.g., FIG. 13) or may be different (e.g., FIGS. 14 and 15). The period for monitoring the movement signal of the magnetic particles may be constant (e.g., FIGS. 13, 14, and 15) or may be altered (e.g., FIG. 16). The period for monitoring the movement signal of the magnetic particles and the period for computing the ratio of the movement signals of the magnetic particles may be constant (e.g., FIGS. 13, 14, and 15) or may be altered (e.g., FIG. 17). The ratio of the movement signals of the magnetic particles may be computed continuously (e.g., FIGS. 13 and 14) or intermittently (e.g., FIG. 15). Alternatively, the ratio of the movement signals may be computed continuously and then intermittently (e.g., FIG. 18) or the same may be computed intermittently and then continuously (e.g., FIG. 19). Various periods for monitoring the movement signal of the magnetic particles, various periods for computing the ratio of the movement signals of the magnetic particles, and various time intervals for computing the signal ratio of magnetic particles can be employed. However, it is preferable that the conditions to prepare a calibration curve and the conditions under which the sample is measured are the same conditions. Other various embodiments which become apparent from descriptions herein also encompassed by the present disclosure.

The fibrinogen determination method of a citrated plasma sample which utilizes said clotting time is not particularly limited. A representative example is as follows. First, 3 types of citrated plasma samples with known but different fibrinogen concentrations are measured by the method described above, the clotting times corresponding to the citrated plasma samples are obtained, and a calibration curve is prepared based thereon in advance. Subsequently, a citrated plasma sample is measured by the method described above, the clotting time is obtained, and the fibrinogen concentration in the citrated plasma sample is determined using the calibration curve prepared above. The calibration curve used in such method may preferably be a linear regression calibration curve with the Y axis indicating LN (fibrinogen concentration) and the X axis indicating LN (clotting time). The determined linear regression is a linear formula (Y=A×X+B), and the fibrinogen concentration in the citrated plasma sample is computed based on the slope of the linear formula (A) and the intercept (B) with the formula shown below.

Fibrinogen concentration in the citrated plasma sample=e^{B}×(clotting time)^{A} [Formula 2]

An example of an apparatus that can be used for fibrinogen determination involving the use of the fibrinogen measurement dry reagent disclosed herein is the CG02N blood coagulation analyzer (A&T Corporation). This apparatus can be operated by designating the point attenuated by 30% from the peak value of the movement signal of the magnetic particles detected at or after the starting point (the starting point of the coagulation reaction) as the clotting point and designating the period from the starting point (the starting point of the coagulation reaction) to the clotting point as the clotting time. The ratio of the movement signals of the magnetic particles can be computed continuously at given time intervals and the starting point can be designated as the first point of the interval during which the ratio is maintained within a given range for a given period of time.

In general, the fibrinogen concentration in a sample is expressed as the fibrinogen concentration in citrated plasma. Since whole blood samples comprise blood cell components in addition to plasma components, it is necessary to take the hematocrit value of the sample into consideration when performing fibrinogen determination on whole blood samples. That is, when using a whole blood sample, it is necessary to subject the fibrinogen concentration converted from the clotting time determined by whole blood measurement to hematocrit correction in order to determine the fibrinogen concentration of the sample. In the case of citrated whole blood, it is necessary to add 9 volumes of whole blood to 1 volume of a sodium citrate solution and mix them with each other to obtain a measurement sample. In contrast, in the case of a heparinized whole blood, a measurement sample is obtained by adding whole blood to heparin sodium or heparin lithium powder and mixing them with each other. As such, the hematocrit correction formula adopted in the case of citrated whole blood is different from that adopted in the case of heparinized whole blood. In the case of citrated whole blood, specifically, the fibrinogen concentration in the sample is computed with the correction formula below.

Fibrinogen concentration in sample=fibrinogen concentration in citrated whole blood×(100/(100−hematocrit value×0.9)) [Formula 3]

When heparinized whole blood is used as the sample, the fibrinogen concentration in the sample is computed with the correction formula below.

Fibrinogen concentration in sample=fibrinogen concentration in heparinized whole blood×0.9×(100/(100−hematocrit value)) [Formula 4]

Incidentally, when citrated whole blood is used as the measurement sample, and the hematocrit value is determined using citrated whole blood, the fibrinogen concentration in the sample is computed with the correction formula below.

Fibrinogen concentration in sample=Fibrinogen concentration in citrated whole blood×(100/(100−hematocrit value)) [Formula 5]

Incidentally, if whole blood is filtered through a filter or a filter medium that does not substantially adsorb fibrinogen, plasma that is suitable for fibrinogen determination can be obtained in a simple manner without using a centrifuge. Use of a plasma sample thus obtained enables accurate and simple fibrinogen concentration determination without the need to perform the five corrections using the correction formulae disclosed above.

The results of fibrinogen determination disclosed herein is extremely consistent with the results of fibrinogen determination by the conventional Clauss method. In addition, the method of the present disclosure yields good reproducibility, and reliable determination can be carried out even when using undiluted whole blood as the sample. Further, reliable determination can be carried out when using undiluted plasma as the sample.

With regard to undiluted whole blood samples (analytes), as described above, the fibrinogen concentration in whole blood can be determined.

Subsequently, the hematocrit value of the undiluted citrated whole blood sample is determined based on the point at which the movement level of the magnetic particles becomes the highest (i.e., the peak point of the waveform) in the manner described below. For convenience of description, the hematocrit value determined based on the peak point of the waveform is referred to as the waveform hematocrit value (waveform Ht value) or the waveform-derived hematocrit value (waveform-derived Ht value) herein. For convenience of description, the hematocrit value determined by the conventional method of measurement is referred to as the measured hematocrit value (measured Ht value) or the directly measured hematocrit value (directly measured Ht value). Subsequently, hematocrit correction can be carried out on the fibrinogen concentration in whole blood using the waveform Ht value to compute the fibrinogen concentration in plasma. Such hematocrit correction may be referred to as “waveform hematocrit correction” herein.

First, the movement signal of magnetic particles is measured for the undiluted citrated whole blood sample. Next, from the measurement data, the clotting time, the measured Ht value (the value obtained by directly measuring the sample by a conventional method), the fibrinogen concentration in whole blood, the fibrinogen concentration in plasma subjected to hematocrit correction using the measured Ht value (this may be referred to as the fibrinogen concentration in plasma determined by the conventional method or the fibrinogen concentration in plasma (conventional value) herein for convenience of description), and the clotting waveform of the movement signals (in particular, the peak value of the waveform) are extracted.

Subsequently, the correlational chart showing the measured Ht values and the peak values of the waveform is prepared. Then, the correlation and the approximation formula are prepared based on the correlational chart. In one embodiment, the approximation formula may be a linear regression formula. In another embodiment, the approximation formula may be a non-linear approximation formula. In another embodiment, the approximation formula can include an exponential function or a logarithmic function. Concerning the approximation formula, reference may be made to general educational material such as “Data Collection and Summary-Statistics and Chemometrics for Analytical Chemistry” (Kyoritsu Shuppan Co., Ltd., 2004) (the original: Jane C. Miller & James N. Miller, Statistics for Analytical Chemistry, 3rd edition). Subsequently, the hematocrit value is computed based on the peak value of the waveform using the approximation formula indicated above (e.g., a linear regression formula); i.e., the waveform Ht value is computed. Subsequently, the correlational chart showing the measured Ht values and the waveform Ht values may be prepared and evaluated. Subsequently, the fibrinogen concentration in whole blood is subjected to hematocrit correction using the waveform Ht values and the fibrinogen concentration in plasma is computed. For convenience of description, the fibrinogen concentration in plasma corrected from the fibrinogen concentration in whole blood with the waveform Ht value is referred to herein as the fibrinogen concentration in plasma determined by the method of the present disclosure or the fibrinogen concentration in plasma of the present disclosure (novel method). Subsequently, the fibrinogen concentration in plasma (conventional value) and the fibrinogen concentration in plasma of the present disclosure (novel method) are plotted to prepare a correlational chart, and the effectiveness of the method of the present disclosure can be verified. The effectiveness of the method of the present disclosure was demonstrated in the examples. As such, it is not necessary to compare the fibrinogen concentration in plasma (conventional value) and the fibrinogen concentration in plasma of the present disclosure (novel method) in each measurement. If the effectiveness of the present disclosure is verified for a certain reagent or apparatus, comparison with the conventional method may be omitted.

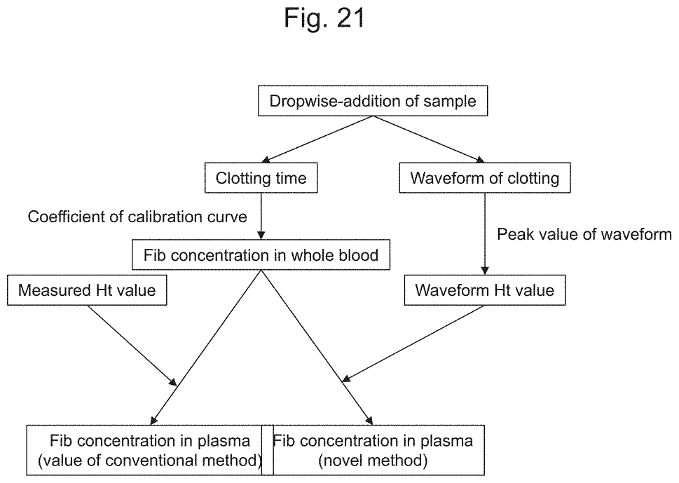

Differences between the method of the present disclosure and the conventional method are shown in the flow chart in FIG. 21. According to the conventional method, it was necessary to measure the hematocrit value using another measurement reagent and another apparatus after the fibrinogen concentration in whole blood was determined. Then, the fibrinogen concentration in whole blood determined using the measured Ht value was subjected to hematocrit correction to compute the fibrinogen concentration in plasma. According to the method of the present disclosure, it is not necessary to use another reagent or apparatus for hematocrit value measurement. After the fibrinogen concentration in whole blood is determined, the waveform Ht value based on the peak value of the waveform derived from the movement level of the magnetic particles is used to subject the fibrinogen concentration in whole blood to waveform hematocrit correction. The fibrinogen concentration in plasma can thus be computed.

According to the present disclosure, fibrinogen can be determined (quantified) rapidly and accurately without the need for preparing reagents or carrying out diluting procedures on the sample. Further, according to the present disclosure, the fibrinogen concentration in plasma (novel method) can be computed by determining the waveform hematocrit value based on the peak value of the waveform of magnetic particles, and subjecting the fibrinogen concentration in whole blood to waveform hematocrit correction based on the waveform hematocrit value without using a specialized reagent for hematocrit value measurement and without performing hematocrit value measurement using such reagent. The present disclosure provides the fibrinogen determination method and an apparatus therefor that can be used in the perinatal period and in the perioperative period. Without limitation, specifically, the fibrinogen determination method and the apparatus disclosed herein can be used for patients in the perinatal period. Further, without limitation, the fibrinogen determination method and the apparatus disclosed herein can be used for patients in perioperative period. The phrase “perinatal period” used herein refers to a period from the 22nd week of pregnancy to before 7 days from birth. This is a definition in accordance with the definition of “the perinatal period” of the International Classification of Diseases, 10th Revision. Further, the phrase “perioperative period” used herein refers to a period including the 3 phases necessary for surgery; i.e., preoperative, intra-operative, and postoperative phases.

### EXAMPLES

The present invention has been described in general terms above. However, the present invention can be further understood with reference to the specific examples below. It should be noted that the examples presented here are provided solely for illustrative purposes and do not limit the scope of the present invention including those described in the claims.

### Preliminary Experiment 1: Correlation Between Fibrinogen Concentration in Plasma and Clotting Time

A 40 mM HEPES buffer (pH 7.35) supplemented with 10 mM CaCl₂·2H₂O, 2.0 (wt/v) % glycine, 80 μg/ml polybrene, 1.2 mg/ml bovine serum albumin, 0.005 (wt/v) % sorbitan monolaurate, and 150 μg/ml GPRP-amide was added to a lyophilized bovine thrombin (Oriental Yeast Co., Ltd.) and dissolved to obtain a reagent solution having 300 NIHU/ml of thrombin activity. To 35 ml of the reagent solution, 0.47 g of triiron tetraoxide (product name: AAT-03; average particle diameter: 0.35 μm; Toda Kogyo Corp.) was added and suspended to obtain a final solution. The final solution (25 μl) was dispensed onto the reaction slide shown in FIG. 1. The reaction slide was stored and frozen in a freezer maintained at −40° C. for one whole day and night. Subsequently, the frozen reaction slide was lyophilized. Lyophilization was performed under the conditions in which the temperature was linearly raised from −30° C. to −20° C. over a period of 24 hours in vacuum, the temperature was linearly raised from −20° C. to 30° C. over a period of 20 hours, the temperature was maintained at 30° C. for 3 hours, and dry air was then applied to release the vacuum. The lyophilized reagent was immediately sealed with an aluminum film in a dehumidified environment.

The method for examining the correlation between the fibrinogen concentration in plasma and the clotting time was carried out in the manner described below. First, human plasma containing 299 mg/dl of fibrinogen and fibrinogen-deficient plasma (Clinisys Associate, Ltd.) were used to prepare 6 serial dilution samples of human plasma from 48 to 299 mg/dl. Subsequently, the lyophilized reagent was mounted on the CG02N blood coagulation analyzer (A&T Corporation), 25 μl each of the serial dilution samples were added thereto, and the clotting time of each sample was determined. In the end, the data were plotted by setting the Y axis to LN (fibrinogen concentration) and the X axis to LN (clotting time), and whether or not linearity could be observed in the prepared chart was examined to inspect whether there was a correlation or not.

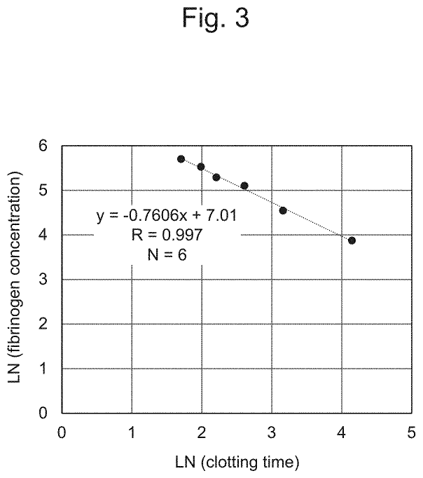

FIG. 3 shows the correlation between the fibrinogen concentration in plasma and the clotting time. As is apparent from FIG. 3, the clotting time was extremely well-correlated with the fibrinogen concentration in the sample.

### Preliminary Experiment 2: Specificity and Reproducibility of Fibrinogen Concentration in Plasma

As the fibrinogen measurement dry reagent, the lyophilized reagent of Preliminary Experiment 1 was used and as the apparatus for fibrinogen determination, the CG02N blood coagulation analyzer (A&T Corporation) was used to examine specificity and reproducibility of the fibrinogen concentration in plasma.

The reagent was mounted on the CG02N analyzer, and 25 μl of a plasma sample with known fibrinogen concentration was added thereto to determine the clotting time. 4 types of plasma samples were each subjected to the procedure 5 times. According to the results obtained in Preliminary Experiment 1, the calibration curve of the lyophilized reagent indicates LN (fibrinogen concentration)=−0.7606×LN (clotting time)+7.01. Thus, the obtained clotting time was converted to the fibrinogen concentration with the formula below.

Fibrinogen concentration in citrated plasma=e^{7.01}×(clotting time)^{-0.7606} [Formula 6]

Specificity was evaluated based on the recovery rate relative to the known fibrinogen concentration, and reproducibility was evaluated based on the CV value (coefficient of variation) obtained by 5 continuous measurements.

The results are shown in Table 2. As is apparent from Table 2, specificity and reproducibility were observed in the fibrinogen concentration.

TABLE 2

Fib concentration

| | 31 | 46 | 107 | 140 |
|---|---|---|---|---|
| Number of assays | mg/dl | mg/dl | mg/dl | mg/dl |
| First | 34 | 48 | 106 | 158 |
| Second | 32 | 49 | 104 | 136 |
| Third | 33 | 51 | 106 | 140 |
| Fourth | 35 | 60 | 95 | 155 |
| Fifth | 32 | 50 | 99 | 133 |
| Average (mg/dl) | 33 | 52 | 102 | 144 |
| Specificity (%) | 107 | 112 | 95 | 103 |
| CV (%) | 4.4 | 9.0 | 4.7 | 8.0 |

### Preliminary Experiment 3: Correlation Between the Clauss Method and the Method Using the Fibrinogen Measurement Dry Reagent According to the Present Disclosure

The correlation between the results of fibrinogen determination by the Clauss method and the results of fibrinogen determination using the fibrinogen measurement dry reagent according to the present disclosure was examined using 51 human plasma samples. Fibrinogen determination by the Clauss method was performed using the Data Fi fibrinogen reagent (Sysmex Corporation) and the KC4 Delta™ coagulation analyzer (Tcoag Ireland Ltd.) by the method described in the package insert attached to the Data Fi fibrinogen reagent.

Fibrinogen determination with the fibrinogen measurement dry reagent was performed with the lyophilized reagent of Preliminary Experiment 1 as the fibrinogen measurement dry reagent and the CG02N blood coagulation analyzer (A&T Corporation) as the apparatus for determining fibrinogen.

The lyophilized reagent was mounted on the CG02N analyzer, 25 μl of the samples was added thereto, and the clotting time of each sample was obtained by the method described above. The obtained clotting time was converted to the fibrinogen concentration using Formula 5.

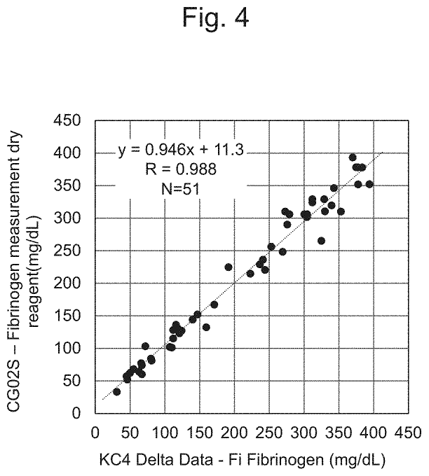

FIG. 4 shows the correlation between the quantified fibrinogen value determined by the Clauss method and the quantified fibrinogen value determined with the fibrinogen measurement dry reagent disclosed herein. As is apparent from FIG. 4, the quantified fibrinogen value determined with the fibrinogen measurement dry reagent disclosed herein is very consistent and highly correlated with the quantified fibrinogen value determined by the Clauss method.

### Preliminary Experiment 4: Correlation Between Citrated Plasma Samples and Citrated Whole Blood Samples

51 citrated whole blood samples were subjected to fibrinogen determination with the fibrinogen measurement dry reagent disclosed herein, 51 citrated plasma samples obtained via centrifugation of the 51 citrated whole blood samples were subjected to fibrinogen determination with the fibrinogen measurement dry reagent disclosed herein, and the correlation between these results of fibrinogen determination was examined. The composition of the fibrinogen measurement dry reagent disclosed herein was as follows:

160 μg/ml polybrene

2.5 (wt/v) % glycine

10 mM CaCl₂·2H₂O

1.2 mg/ml bovine serum albumin

0.005 (wt/v) % sorbitan monolaurate

200 μg/ml GPRP-amide

40 mM HEPES-NaOH buffer (pH 7.35)

333 NIHU/ml bovine thrombin

The apparatus and the procedure employed herein were identical to those in Preliminary Experiment 3. Since the calibration curve of the lyophilized reagent indicates: LN (fibrinogen concentration)=−0.7636×LN (clotting time)+7.22, the determined clotting time was converted to the fibrinogen concentration with the formula below.

Fibrinogen concentration in citrated plasma=e^{7.22}×(clotting time)^{-0.7636} [Formula 7]

When citrated whole blood was used as the measurement sample, the fibrinogen concentration in the sample was determined in the following manner. First, hematocrit values of the 51 citrated whole blood samples were determined using the blood cell counter MYTHIC22 (J) (A&T Corporation). Subsequently, the lyophilized reagent was mounted on the CG02N blood coagulation analyzer (A&T Corporation), the assay mode was changed to the whole blood assay mode, 25 μl of the citrated whole blood was added thereto, and the clotting time of each sample was then determined.

The clotting time was converted to the fibrinogen concentration using Formula 7, and the fibrinogen concentration in the citrated whole blood sample was determined using Formula 5.

When citrated plasma was used as the measurement sample, the fibrinogen concentration of the sample was determined in the manner described below. First, 51 citrated whole blood samples were centrifuged at 4° C. and 3,000 rpm for 15 minutes, and 51 citrated plasma samples were obtained from the supernatant. Subsequently, the lyophilized reagent above was mounted on the CG02N analyzer, the assay mode was changed to the plasma assay mode, 25 μl of the citrated plasma was added, and the clotting time of each sample was determined. The clotting time was converted to the fibrinogen concentration using Formula 7.

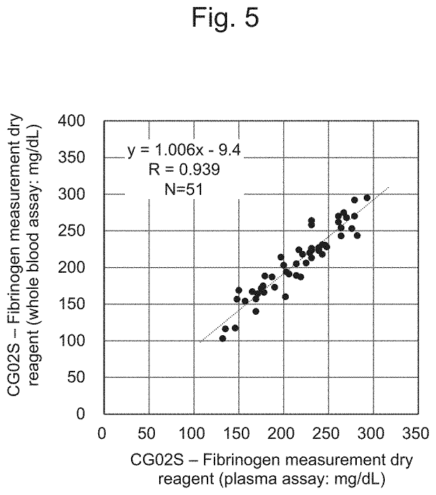

FIG. 5 shows the correlation between the quantified fibrinogen value in the citrated plasma measurement samples and the quantified fibrinogen value in the citrated whole blood measurement samples determined with the fibrinogen measurement dry reagent disclosed herein. As is apparent from FIG. 5, the quantified fibrinogen value in the citrated whole blood sample is very consistent and highly correlated with the quantified fibrinogen value in the citrated plasma samples determined with the fibrinogen measurement dry reagent disclosed herein.

### Preliminary Experiment 5: Preparation of Reagents at Various Glycine Concentrations and Evaluation Thereof

The effects of the glycine content in the fibrinogen measurement dry reagent were examined in terms of the clotting time of the citrated plasma, the clotting time of the citrated whole blood, and simultaneous reproducibility thereof. First, the reagent composition as used in Preliminary Experiment 4 was used to prepare lyophilized reagents, although the glycine concentration in the reagent composition was set as 0.5%, 1.0%, 1.5%, 2.0%, 2.5%, 3.0%, 3.5%, 4.0%, 4.5%, and 5.0% for each sample. Subsequently, citrated plasma with a fibrinogen concentration of 181 mg/dl was repeatedly measured using the lyophilized reagents on the CG02N analyzer, and the clotting time and the CV values obtained by the 5 repeated measurements were recorded.

TABLE 3

| | Present disclosure | | | | | | | | | |
|---|---|---|---|---|---|---|---|---|---|---|
| | Glycine concentration | | | | | | | | | |
| Plasma | 0.5% | 1.0% | 1.5% | 2.0% | 2.5% | 3.0% | 3.5% | 4.0% | 4.5% | 5.0% |
| 1 (s) | 13.9 | 13.4 | 11.1 | 10.7 | 9.4 | 7.5 | 6.2 | 6.5 | 6.0 | 4.4 |
| 2 (s) | 14.5 | 14.7 | 10.7 | 10.4 | 9.8 | 6.4 | 7.4 | 6.6 | 5.7 | 4.7 |
| 3 (s) | 13.9 | 15.3 | 10.8 | 10.9 | 8.6 | 8.4 | 6.2 | 6.8 | 4.9 | 4.5 |
| 4 (s) | 14.7 | 13.8 | 10.6 | 11.2 | 10.3 | 7.4 | 7.2 | 7.1 | 6.0 | 5.1 |
| 5 (s) | 12.6 | 15.1 | 11.2 | 9.7 | 9.2 | 7.2 | 8.0 | 6.7 | 5.5 | 5.1 |
| AVG (s) | 13.9 | 14.5 | 10.9 | 10.6 | 9.5 | 7.4 | 7.0 | 6.7 | 5.6 | 4.8 |
| SD | 0.8 | 0.8 | 0.3 | 0.6 | 0.6 | 0.7 | 0.8 | 0.2 | 0.5 | 0.3 |
| CV (%) | 5.9 | 5.7 | 2.4 | 5.4 | 6.8 | 9.7 | 11.2 | 3.4 | 8.1 | 6.9 |

(s): Clotting time;
AVG: average;
SD: standard deviation;
CV: Coefficient of variation

As shown in Table 3, when the glycine concentration in the reagent is less than 1.5%, the clotting time is extremely prolonged because of the lack of reagent solubility. However, when the glycine concentration in the reagent is 1.5% or higher, solubility is enhanced, and a shortened clotting time is obtained. When the glycine concentration in the reagent is over 4.5%, the clotting time determined by the CG02N blood coagulation analyzer is shorter than the lower detection limit, which is 5.0 seconds. This indicates that it is not possible to perform fibrinogen quantification of a sample with a fibrinogen concentration exceeding 181 mg/dl. In other words, in the case of a reagent with the glycine concentration exceeding 4.5%, it is not possible to determine whether or not the fibrinogen concentration in the sample has returned within the normal range (200 to 400 mg/dl) as a result of administration of a fibrinogen preparation. Therefore, in the case of plasma measurements, it is apparent that the glycine concentration in the reagent is preferably within the range of 1.5% to 4.0%.

Subsequently, citrated whole blood samples with a fibrinogen concentration of 181 mg/dl were repeatedly measured 5 times using the lyophilized reagents on the CG02N analyzer, and the clotting time and the CV values obtained by the 5 repeated measurements were recorded.

TABLE 4

| | Present disclosure | | | | | | | | | |
|---|---|---|---|---|---|---|---|---|---|---|
| | Glycine concentration | | | | | | | | | |
| Whole blood | 0.5% | 1.0% | 1.5% | 2.0% | 2.5% | 3.0% | 3.5% | 4.0% | 4.5% | 5.0% |
| 1 (s) | 46.9 | 52.1 | 30.6 | 25.3 | 20.7 | 17.3 | 15.4 | 12.2 | 10.2 | 5.6 |
| 2 (s) | 39.4 | 49.5 | 32.6 | 24.1 | 18.7 | 18.5 | 14.2 | 12.1 | 12.4 | 6.9 |
| 3 (s) | 39.3 | 58.9 | 27.7 | 27.7 | 22.4 | 16.2 | 15.2 | 11.9 | 10.3 | 7.3 |
| 4 (s) | 58.0 | 65.5 | 34.5 | 23.2 | 22.5 | 17.3 | 16.0 | 9.8 | 11.4 | 8.1 |
| 5 (s) | 47.2 | 55.1 | 37.8 | 26.4 | 22.1 | 14.9 | 15.0 | 9.0 | 11.1 | 9.0 |
| AVG (s) | 46.2 | 56.2 | 32.6 | 25.3 | 21.3 | 16.8 | 15.2 | 11.0 | 11.1 | 7.4 |
| SD | 7.7 | 6.3 | 3.8 | 1.8 | 1.6 | 1.4 | 0.7 | 1.5 | 0.9 | 1.3 |
| CV (%) | 16.6 | 11.1 | 11.7 | 7.1 | 7.6 | 8.1 | 4.3 | 13.6 | 8.1 | 17.3 |

(s): Clotting time;
AVG: average;
SD: standard deviation;
CV: Coefficient of variation

As shown in Table 4, the clotting time is extremely prolonged because of a lack of reagent solubility when the glycine concentration in the reagent is less than 1.5%. On the other hand, when the glycine concentration in the reagent is 1.5% or higher, solubility is enhanced, and a shortened clotting time is obtained. In the case of whole blood measurements, accordingly, it is apparent that the glycine concentration in the reagent is preferably 1.5% or higher.

### Comparative Example 1: Comparison of Properties with Lyophilized Reagent of Conventional Composition

Properties of the fibrinogen measurement dry reagent disclosed herein were compared with those of a lyophilized reagent prepared with the reagent composition described in JP Patent No. 3469909.

A fibrinogen measurement dry reagent with the glycine concentration of 2.5% was prepared by the method described in Preliminary Experiment 1. Also, a lyophilized reagent having the composition described below was prepared by the method described in Preliminary Experiment 1. The reagent composition is reported in JP H05-219993 A (JP Patent No. 3469909).

Reagent composition of Comparative Example:

15 μg/ml polybrene

10 mM CaCl₂·2H₂O

1.0 (wt/v) % bovine serum albumin

0.08 (wt/v) % Polyethylene glycol 6000

200 μg/ml polymerization inhibitor (GPRP-amide)

50 mM Tris-HCl buffer (pH8.0)

50 IU/ml bovine thrombin

110 mM NaCl

The citrated plasma samples and the citrated whole blood samples with the fibrinogen concentration of 162 mg/dl were repeatedly measured 5 times using the relevant reagents on the CG02N analyzer, and the clotting time and the CV values obtained by the 5 repeated measurements were recorded. Also, changes in the movement signal of the magnetic particles detected with the elapse of time in measurements were recorded.

TABLE 5

| | Plasma assay | | Whole blood assay | |
|---|---|---|---|---|
| | Dry reagent | Lyophilized | Dry reagent | Lyophilized |
| | for fibrinogen | reagent of | for fibrinogen | reagent of |
| Number | determination of the | conventional | determination of the | conventional |
| of assays | present disclosure | composition | present disclosure | composition |
| First (sec) | 10.5 | 31.6 | 25.6 | 54.1 |
| Second (sec) | 9.8 | 32.4 | 24.4 | 45.7 |
| Third (sec) | 11.1 | 35.4 | 24.0 | 62.9 |
| Fourth (sec) | 11.0 | 29.8 | 23.4 | 54.2 |
| Fifth (sec) | 11.1 | 32.6 | 24.8 | 77.4 |
| Average (sec) | 10.7 | 32.4 | 24.4 | 58.9 |
| Standard deviation | 0.6 | 2.0 | 0.8 | 12.0 |
| CV (%) | 5.2 | 6.3 | 3.4 | 20.4 |

CV: Coefficient of variation

As shown in Table 5, it is clear that the clotting time obtained with the fibrinogen measurement dry reagent disclosed herein is shorter than that obtained with a lyophilized reagent of a conventional composition, and accordingly, the reproducibility of the clotting time is good.

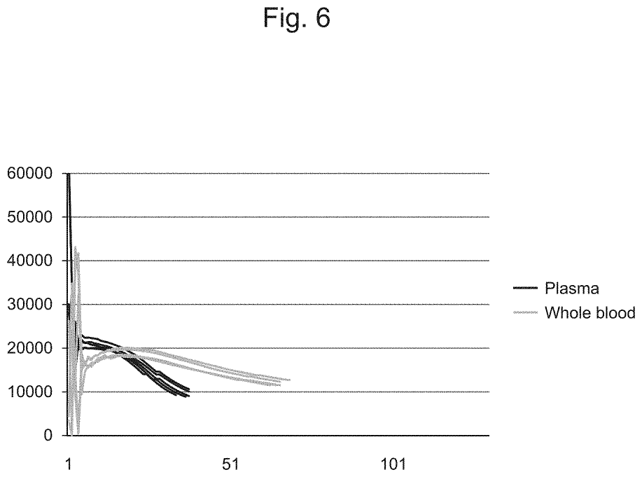

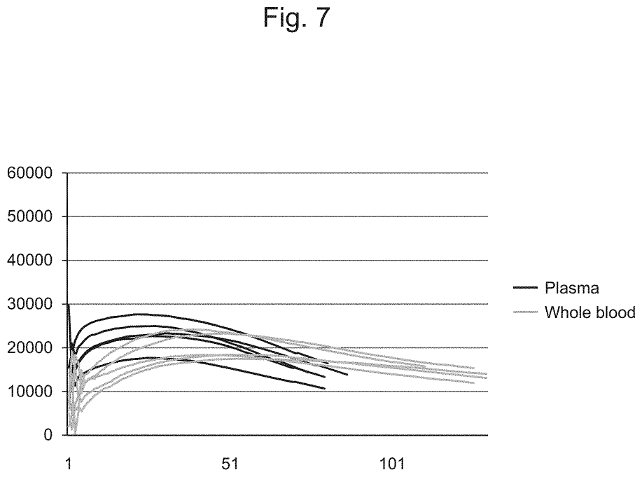

FIG. 6 and FIG. 7 show changes in the movement signal of the magnetic particles detected with the elapse of time in the measurements. FIG. 6 shows a chart demonstrating changes in the movement signal of the magnetic particles with the elapse of time when measured with the fibrinogen measurement dry reagent disclosed herein. FIG. 7 shows a chart demonstrating changes in the movement signal of the magnetic particles with the elapse of time when measured with the lyophilized reagent prepared with the reagent composition of the conventional technique. In the charts, the horizontal axis indicates the time elapsed after the sample is added, a numerical value “51” indicates 25.5 seconds, and a numerical value “101” indicates 50.5 seconds. The vertical axis indicates the amount of change in scattered light; i.e., the movement signal of the magnetic particles (unit: counts). Changes in the movement signal of the magnetic particles with the elapse of time were more constant among the 5 measurements conducted with the fibrinogen measurement dry reagent disclosed herein and it is clear that the movement signal of the magnetic particles are attenuated to a significant extent as the clotting reaction proceeds. In contrast, changes in the movement signal of the magnetic particles with the elapse of time varied significantly among the 5 measurements conducted with the lyophilized reagent of the conventional composition, and the attenuation of the movement signal of the magnetic particles as the clotting reaction proceeds is moderate. When such reagent is used, there is a risk of erroneous measurement.

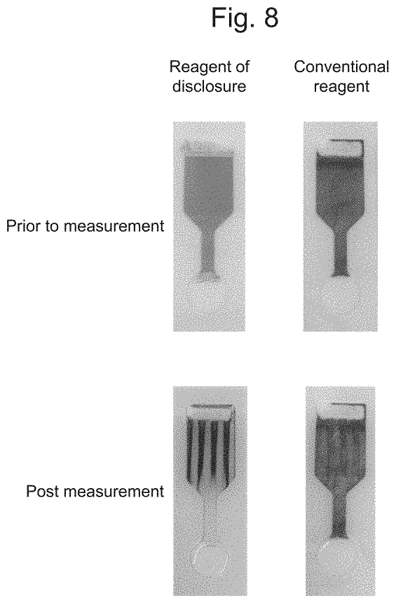

FIG. 8 shows photographs of the reagents before and after plasma measurements. In FIG. 8, the upper photographs show reagents before the measurements and the lower photographs show the reagents after the measurements. In the case of the lyophilized reagent of the conventional composition, reagent solubility is insufficient. Therefore, magnetic particles are aggregated locally after the measurements, and it is difficult to identity the magnetic particle lines (beams) derived from the permanent magnetic field. This means that the movement of magnetic particles does not necessarily correspond to the change in the viscosity in the reaction system caused as the coagulation reaction proceeds. In contrast, in the case of the reagent disclosed herein (glycine concentration in the reagent: 2.5%), reagent solubility is improved, and it is possible to clearly identify the magnetic particle lines derived from the permanent magnetic field. In the same manner, it was possible to clearly identify the magnetic particle lines derived from the permanent magnetic field with the reagent of the present disclosure with the glycine concentration of 1.5%, 2.0%, 3.0%, 3.5%, or 4.0%. Concerning the reagents with the glycine concentration of 4.5% and 5.0%, local polymerization of magnetic particles was observed and the appearance of the particles was not always good after the measurements.

### Preliminary Experiment 6: Measurement of Clotting Time by the Fibrinogen Determination Method Disclosed Herein

First, in accordance with Preliminary Experiment 1, the fibrinogen measurement dry reagent was prepared in the manner described below.

A 40 mM HEPES buffer (pH 7.35) supplemented with 10 mM CaCl₂·2H₂O, 2.0 (wt/v) % glycine, 160 μg/ml polybrene, 1.2 mg/ml bovine serum albumin, 0.005 (wt/v) % sorbitan monolaurate, and 200 μg/ml GPRP-amide was added to a lyophilized bovine thrombin product (Oriental Yeast Co., Ltd.) and dissolved to obtain a reagent solution having 333 NIHU/ml of thrombin activity. To 35 ml of the reagent solution, 0.47 g of triiron tetraoxide (product name: AAT-03; average particle diameter: 0.35 μm; Toda Kogyo Corp.) was added and suspended to obtain a final solution. The final solution (25 μl) was dispensed onto the reaction slide shown in FIG. 1. The reaction slide was stored and frozen in a freezer maintained at −40° C. for one whole day and night. Subsequently, the frozen reaction slide was lyophilized. Lyophilization was performed under the conditions in which the temperature was linearly raised from −30° C. to −20° C. over a period of 24 hours in vacuum, the temperature was linearly raised from −20° C. to 30° C. over a period of 20 hours, the temperature was maintained at 30° C. for 3 hours, and dry air was then applied to release the vacuum. The lyophilized reagent was immediately sealed with an aluminum film in a dehumidified environment.

Whole blood samples were measured using the fibrinogen measurement dry reagent described above by the fibrinogen determination method disclosed herein. According to this method, monitoring of the movement signal of the magnetic particles was initiated immediately after the samples were added and monitoring was performed at intervals of 0.5 seconds. That is, the period for monitoring the movement signal of the magnetic particles is 0.5 seconds. The ratio of the movement signals of the magnetic particles was then continuously computed at intervals of 1 second. In other words, the time interval used to compute the ratio of the movement signals of the magnetic particles is 1 second. That is, the ratio of the movement signals of the magnetic particles is computed as follows: (the movement signal of the magnetic particles detected at the monitoring time of 1.0 second)/(the movement signal of the magnetic particles detected at the monitoring time of 0 seconds), (the movement signal of the magnetic particles detected at the monitoring time of 1.5 seconds)/(the movement signal of the magnetic particles detected at the monitoring time of 0.5 seconds), (the movement signal of the magnetic particles detected at the monitoring time of 2.0 seconds)/(the movement signal of the magnetic particles detected at the monitoring time of 1.0 second) . . . . The interval during which the ratio was maintained within a range of 1.0±0.1 for 1.5 seconds was the period of 5.0 to 6.5 seconds in terms of the monitoring time. The first point thereof was at the monitoring time of 5.0 seconds, and this point was thus designated as the starting point (the starting point of the coagulation reaction, measurement time 0 (sec)). The peak value of the movement signal of the magnetic particles at or after the starting point was 2726c detected at the monitoring time of 7.0 seconds. The movement signal of the magnetic particles that was lower by 30% from the peak value of the movement signal of the magnetic particles at or after the starting point was computed to be 1908c. That is, the end point was the point at which the movement signal of the magnetic particles was 1908c, and the clotting time was computed to be 20.1 seconds. The results are shown in Table 6.

TABLE 6

| | | Ratio of movement signal of magnetic | | |
|---|---|---|---|---|
| Time point for monitoring | Movement signal of | particles at a time interval of 1 sec | Start/ | Measurement |
| the movement signals of | magnetic particles | for computing the ratio of movement | peak/ | time |
| magnetic particles (sec) | (C) | signals of magnetic particles (—) | end | (sec) |
| 0 | 1430 | — | | |
| 0.5 | 86 | — | | |
| 1.0 | 359 | 0.25 | | |
| 1.5 | 114 | 1.33 | | |
| 2.0 | 3722 | 10.37 | | |
| 2.5 | 4235 | 37.15 | | |
| 3.0 | 1841 | 0.49 | | |
| 3.5 | 3534 | 0.83 | | |
| 4.0 | 2890 | 1.57 | | |
| 4.5 | 2389 | 0.68 | | |
| 5.0 | 2673 | 0.92 | Start | 0 |
| 5.5 | 2581 | 1.08 | | 0.5 |
| 6.0 | 2682 | 1.00 | | 1.0 |
| 6.5 | 2651 | 1.03 | | 1.5 |
| 7.0 | 2726 | 1.02 | Peak | 2.0 |
| 7.5 | 2678 | 1.01 | | 2.5 |
| 8.0 | 2721 | 1.00 | | 3.0 |
| 8.5 | 2673 | 1.00 | | 3.5 |
| 8.0 | 2708 | 1.00 | | 4.0 |
| 9.5 | 2665 | 1.00 | | 4.5 |
| 10.0 | 2677 | 0.99 | | 5.5 |
| 10.5 | 2635 | 0.99 | | 6.0 |
| 11.0 | 2635 | 0.98 | | 6.5 |
| . | . | . | | . |
| . | . | . | | . |
| . | . | . | | . |
| 22.0 | 2031 | 0.98 | | 17.5 |
| 22.5 | 2007 | 0.98 | | 18.0 |
| 23.0 | 1980 | 0.97 | | 18.5 |
| 23.5 | 1957 | 0.98 | | 19.0 |
| 24.0 | 1940 | 0.98 | | 19.5 |
| 24.5 | 1913 | 0.98 | | 20.0 |
| — | 1908 | — | End | 20.1 |
| 25.0 | 1889 | 0.97 | | 20.5 |
| 25.5 | 1870 | 0.98 | | 21.0 |

Peak movement signal of magnetic particles at or after the starting point: 2726
Movement signal of magnetic particles attenuated by 30% from the peak movement signal of magnetic particles at or after the starting point: 1908
Clotting time = 20.1 sec

### Preliminary Experiment 7: Comparison of the Conventional Fibrinogen Determination Method (the Determination Method According to JP Patent No. 2980468) with the Fibrinogen Determination Method Disclosed Herein (the Present Disclosure) when Undiluted Whole Blood Samples are Measured Using the Fibrinogen Measurement Dry Reagent

The fibrinogen measurement dry reagent was prepared in the manner described above.

First, the calibration curve according to the conventional determination method (the determination method according to JP Patent No. 2980468) was set up. The calibration curve was set up in the manner described below. Human plasma containing 304 mg/dl of fibrinogen and fibrinogen-deficient plasma (Clinisys Associate, Ltd.) were used to prepare 7 serial dilution samples of human plasma from 37 to 304 mg/dl. Subsequently, the fibrinogen measurement dry reagent was mounted on the CG02N blood coagulation analyzer (A&T Corporation), 25 μl each of the serial dilution samples were added thereto, and the clotting time of each sample was obtained. Finally, the data were plotted by setting the Y axis to LN (fibrinogen concentration) and the X axis to LN (clotting time) and determining the regression formula to compute the calibration curve according to the conventional determination method.

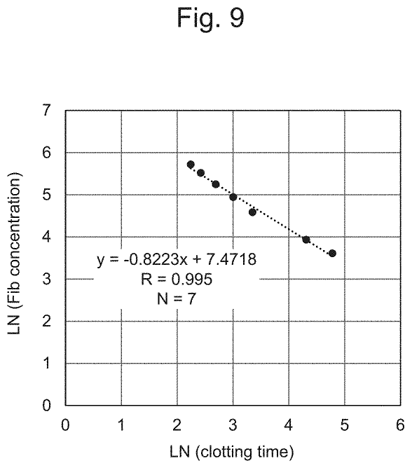

As a result, the calibration curve according to the conventional determination method was found to be as follows (FIG. 9).

LN(fibrinogen concentration)=−0.8223×LN(clotting time)+7.4718 [Formula 8]

Based on the calibration curve formula above, the fibrinogen concentration conversion formula shown below was employed.

Fibrinogen concentration in sample=e^{7.4718}×(clotting time)^{-0.8223} [Formula 9]

Subsequently, the calibration curve according to the determination method disclosed herein was set up. The calibration curve was set up in the manner described below. Human plasma containing 304 mg/dl of fibrinogen and fibrinogen-deficient plasma (Clinisys Associate, Ltd.) were used to prepare 7 serial dilution samples of human plasma from 37 to 304 mg/dl. Subsequently, the fibrinogen measurement dry reagent was mounted on the CG02N blood coagulation analyzer (A&T Corporation), the software disclosed herein was integrated therein, 25 μl each of the serial dilution samples was added thereto, and the clotting time of each sample was determined. Finally, the data were plotted by setting the Y axis to LN (fibrinogen concentration) and the X axis to LN (clotting time) and determining the regression formula to compute the calibration curve according to the determination method disclosed herein.

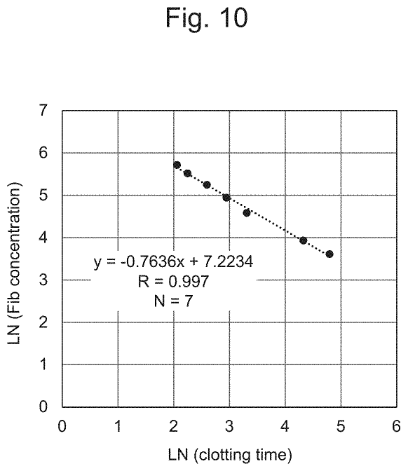

As a result, the calibration curve according to the determination method disclosed herein was found to be as follows (FIG. 10):

LN(fibrinogen concentration)=−0.7636×LN(clotting time)+7.2234 [Formula 10]

Based on the calibration curve formula above, the fibrinogen concentration conversion formula shown below was employed.

Fibrinogen concentration in sample=e^{7.2234}×(clotting time)^{-0.7636} [Formula 11]

Blood samples were obtained from one healthy subject using 7 vacuum blood collection tubes with sodium citrate (2 ml) to obtain 14 ml of citrated whole blood samples. The 7 blood collection tubes were subjected to centrifugation at 4° C. and 3,000 rpm for 15 minutes. Among the 7 centrifuged blood collection tubes, 3 tubes were set aside, and the supernatants (plasma samples) from the 4 blood collection tubes were aliquoted in amounts of 1 ml each and dispensed into PP (polypropylene) containers to obtain 4 ml of citrated plasma sample A. Citrated plasma sample A (2.80 ml) was added to 1 out of the 3 remaining blood collection tubes which were set aside, and the blood collection tube was hermetically sealed, followed by mixing by inversion to obtain citrated whole blood sample B. Separately, citrated plasma sample A (0.40 ml) was added to (another) 1 out of the remaining 3 blood collection tubes set aside, and the collection tube was hermetically sealed, followed by mixing by inversion to obtain citrated whole blood sample C. Further, 0.56 ml of the supernatant (plasma sample) was removed from 1 out of the remaining 3 blood collection tubes set aside, and the collection tube was hermetically sealed, followed by mixing by inversion to obtain citrated whole blood sample D.

The hematocrit values of citrated whole blood sample B, citrated whole blood sample C, and citrated whole blood sample D were determined using the blood cell counter MYTHIC22 (J) (distributed by A&T Corporation). As a result, the hematocrit value of citrated whole blood sample B was 15%, that of citrated whole blood sample C was 30%, and that of citrated whole blood sample D was 50%.

Fibrinogen concentrations in citrated plasma sample A, citrated whole blood sample B, citrated whole blood sample C. and citrated whole blood sample D were examined according to the conventional determination method (JP Patent No. 2980468).

The fibrinogen measurement dry reagent was mounted on the CG02N analyzer, the assay mode was changed to the plasma assay mode, 25 μl of citrated plasma sample A was added, and the clotting time was obtained. This procedure was carried out 5 times. The obtained clotting time was applied to the conversion formula mentioned above (i.e., fibrinogen concentration in sample=e^{7.4718}×(clotting time)^{-0.8223}) and converted to the fibrinogen concentration in citrated plasma sample A according to the conventional determination method.

The fibrinogen measurement dry reagent was mounted on the CG02N analyzer, the assay mode was changed to the whole blood assay mode, 25 μl of citrated whole blood sample B was added thereto, and the clotting time was obtained. This procedure was carried out 5 times. The obtained clotting time was applied to the same conversion formula and converted to the fibrinogen concentration. In addition, the fibrinogen concentration in citrated whole blood sample B according to the conventional determination method was determined with the formula shown below.

Fibrinogen concentration in citrated whole blood sample B according to the conventional determination method=converted fibrinogen concentration×(100/(100 15)) [Formula 12]

The fibrinogen measurement dry reagent was mounted on the CG02N analyzer, the assay mode was changed to the whole blood assay mode, 25 μl of citrated whole blood sample C was added thereto, and the clotting time was obtained. This procedure was carried out 5 times. The obtained clotting time was applied to the same conversion formula and converted to the fibrinogen concentration. In addition, the fibrinogen concentration in citrated whole blood sample C according to the conventional determination method was determined with the formula shown below.

Fibrinogen concentration in citrated whole blood sample C according to the conventional determination method=converted fibrinogen concentration−(100/(100−30)) [Formula 13]

The fibrinogen measurement dry reagent was mounted on the CG02N analyzer, the assay mode was changed to the whole blood assay mode, 25 μl of citrated whole blood sample D was added thereto, and the clotting time was obtained. This procedure was carried out 5 times. The obtained clotting time was applied to the same conversion formula and converted to the fibrinogen concentration. In addition, the fibrinogen concentration in citrated whole blood sample D according to the conventional determination method was determined with the formula shown below.

Fibrinogen concentration in citrated whole blood sample D according to the conventional determination method=converted fibrinogen concentration×(100/(100−50)) [Formula 14]

Subsequently, fibrinogen concentrations in citrated plasma sample A, citrated whole blood sample B, citrated whole blood sample C. and citrated whole blood sample D were examined according to the determination method disclosed herein.

The fibrinogen measurement dry reagent was mounted on the CG02N analyzer, the software that implements the fibrinogen determination method disclosed herein was integrated therein, the assay mode was changed to the plasma assay mode, 25 μl of citrated plasma sample A was added, and the clotting time was obtained. This procedure was carried out 5 times. The obtained clotting time was applied to the conversion formula mentioned above (i.e., fibrinogen concentration in sample=e^{7.2234}×(clotting time)^{-0.7636}) and converted to the fibrinogen concentration in citrated plasma sample A according to the determination method disclosed herein.

The fibrinogen measurement dry reagent was mounted on the CG02N analyzer, the software that implements the fibrinogen determination method disclosed herein was integrated therein, the assay mode was changed to the whole blood assay mode, 25 μl of citrated whole blood sample B was added, and the clotting time was obtained. This procedure was carried out 5 times. The obtained clotting time was applied to the same conversion formula and converted to the fibrinogen concentration. In addition, the fibrinogen concentration in citrated whole blood sample B according to the determination method disclosed herein was determined with the formula shown below.

Fibrinogen concentration in citrated whole blood sample B according to the determination method disclosed herein=converted fibrinogen concentration×(100/(100−15)) [Formula 15]

The fibrinogen measurement dry reagent was mounted on the CG02N analyzer, the software that implements the fibrinogen determination method disclosed herein was integrated therein, the assay mode was changed to the whole blood assay mode, 25 μl of citrated whole blood sample C was added, and the clotting time was obtained. This procedure was carried out 5 times. The obtained clotting time was applied to the same conversion formula and converted to the fibrinogen concentration. In addition, the fibrinogen concentration in citrated whole blood sample C according to the determination method disclosed herein was determined with the formula shown below.

Fibrinogen concentration in citrated whole blood sample C according to the determination method disclosed herein=converted fibrinogen concentration×(100/(100−30)) [Formula 16]

The fibrinogen measurement dry reagent was mounted on the CG02N analyzer, the software that implements the fibrinogen determination method disclosed herein was integrated therein, the assay mode was changed to the whole blood assay mode, 25 μl of citrated whole blood sample D was added, and the clotting time was obtained. This procedure was carried out 5 times. The obtained clotting time was applied to the same conversion formula and converted to the fibrinogen concentration. In addition, the fibrinogen concentration in citrated whole blood sample D according to the determination method disclosed herein was determined with the formula shown below.

Fibrinogen concentration in citrated whole blood sample D according to the determination method disclosed herein=converted fibrinogen concentration×(100/(100−50)) [Formula 17]

In addition, citrated plasma sample A was determined by the Clauss method. Fibrinogen determination by the Clauss method was performed using the Data Fi fibrinogen reagent (Sysmex Corporation) and the KC4 Delta™ coagulation analyzer (Tcoag Ireland Ltd.) by the method described in the package insert attached to the Data Fi fibrinogen reagent. Fibrinogen determination was performed 5 times, and the average value thereof (i.e., 224 mg/dl) was determined to be the fibrinogen concentration in citrated plasma sample A by the Clauss method. The results are shown below.

TABLE 7

| | Fib concentration | | | |
|---|---|---|---|---|
| | | Citrated | Citrated | Citrated |
| | Citrated | whole | whole | whole |
| Number | plasma | blood B Ht | blood C Ht | blood D Ht |
| of assays | A | value = 15% | value = 30% | value = 50% |
| First | 231 | 229 | 229 | 259 |
| Second | 219 | 222 | 239 | 263 |
| Third | 219 | 235 | 241 | 233 |
| Fourth | 223 | 232 | 216 | 278 |
| Fifth | 231 | 219 | 209 | 257 |
| Average (mg/dl) | 225 | 228 | 227 | 258 |
| Specificity (%) | 100.4 | 101.8 | 101.3 | 115.2 |
| CV (%) | 2.7 | 2.9 | 6.2 | 6.2 |

TABLE 8

| | Fib concentration | | | |
|---|---|---|---|---|
| | | Citrated | Citrated | Citrated |
| | Citrated | whole | whole | whole |
| Number | plasma | blood B Ht | blood C Ht | blood D Ht |
| of assays | A | value = 15% | value = 30% | value = 50% |
| First | 235 | 229 | 227 | 220 |
| Second | 223 | 214 | 238 | 234 |
| Third | 212 | 234 | 248 | 216 |
| Fourth | 224 | 232 | 215 | 229 |
| Fifth | 228 | 222 | 208 | 206 |
| Average (mg/dl) | 224 | 226 | 227 | 221 |
| Specificity (%) | 100.0 | 100.9 | 101.3 | 98.7 |
| CV (%) | 3.6 | 3.6 | 7.2 | 5.0 |

Table 7 shows the results of measurements according to the conventional determination method and Table 8 shows the results of measurements according to the determination method disclosed herein. Specificity was evaluated based on the recovery rate relative to the fibrinogen concentration (224 mg/dl) of citrated plasma sample A determined by the Clauss method. A whole blood sample with a higher hematocrit value has higher viscosity. According to Table 7, the whole blood sample D with high viscosity shows higher values than the plasma sample A in all measurements. That is, the results shown in Tables 7 and 8 clearly demonstrate that it is not possible to accurately quantify the fibrinogen concentration of a whole blood sample with a high hematocrit value according to the conventional determination method; however, it is possible to accurately quantify the fibrinogen concentration of a whole blood sample with a high hematocrit value according to the determination method disclosed herein.

### Preliminary Experiment 8: Correlation Between the Quantified Fibrinogen Value Determined by the Clauss Method and the Quantified Fibrinogen Value Determined by the Determination Method Disclosed Herein

The correlation between the results of fibrinogen determination by the Clauss method and the results of fibrinogen determination by the determination method disclosed herein was examined using 104 citrated plasma samples. Fibrinogen determination by the determination method disclosed herein was performed in the manner described below.

The fibrinogen measurement dry reagent was mounted on the CG02N analyzer, the software that implements the fibrinogen determination method disclosed herein was integrated therein, the assay mode was changed to the plasma assay mode, 25 μl of the citrated plasma sample was added, and the clotting time was obtained. The obtained clotting time was applied to the conversion formula mentioned above (i.e., fibrinogen concentration in sample=e^{7.2234}×(clotting time)^{-0.7636}) and converted to the fibrinogen concentration, and the converted fibrinogen concentration was designated as the fibrinogen concentration according to the determination method disclosed herein.

Fibrinogen determination according to the Clauss method was performed using the Hemos IL Fib CXL reagent (LSI Medience Corporation) and the clinical laboratory system STACIA (LSI Medience Corporation). Determination was performed by the method described in the package insert attached to Hemos IL Fib CXL.

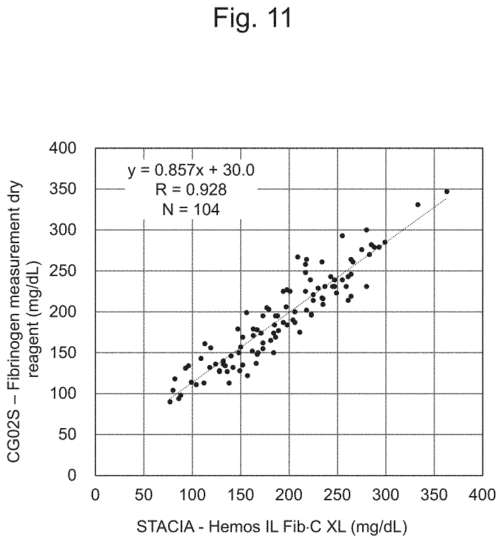

FIG. 11 shows the correlation between the quantified fibrinogen value determined by the Clauss method and the quantified fibrinogen value determined by the determination method disclosed herein. As is apparent from FIG. 11, the quantified fibrinogen value determined by the determination method disclosed herein is very consistent and highly correlated with the quantified fibrinogen value determined by the Clauss method.

### Preliminary Experiment 9: Correlation Between the Quantified Fibrinogen Value Determined Using the Citrated Plasma Sample and the Quantified Fibrinogen Value Determined Using the Citrated Whole Blood Sample by the Method Disclosed Herein

80 citrated whole blood samples were subjected to fibrinogen quantification by the determination method disclosed herein, 80 citrated plasma samples obtained via centrifugation of the same 80 citrated whole blood samples were subjected to fibrinogen determination by the determination method disclosed herein, and the correlation between these results of fibrinogen determination was examined.

First, hematocrit values of the 80 citrated whole blood samples were determined using the blood cell counter MYTHIC22 (J) (A&T Corporation). Subsequently, the fibrinogen measurement dry reagent was mounted on the CG02N analyzer, the software that implements the fibrinogen determination method disclosed herein was integrated therein, the assay mode was changed to the whole blood assay mode, 25 μl of the citrated whole blood sample was added, and the clotting time of each sample was obtained. The obtained clotting time was applied to the conversion formula mentioned above and shown below and converted to the fibrinogen concentration.

Fibrinogen concentration in sample=e^{7.2234}×(clotting time)^{-0.7636} [Formula 18]

Finally, the fibrinogen concentration in the citrated whole blood sample was determined with the formula below.

Fibrinogen concentration in sample=converted fibrinogen concentration×(100/(100−hematocrit value)) [Formula 19]

The 80 citrated whole blood samples for which the above measurement was completed were subjected to centrifugation at 4° C. and 3,000 rpm for 15 minutes, and the supernatant was collected to obtain 80 citrated plasma samples. Subsequently, the fibrinogen measurement dry reagent was mounted on the CG02N analyzer, the software that implements the fibrinogen determination method disclosed herein was integrated therein, the assay mode was changed to the plasma assay mode, 25 μl of the citrated plasma sample was added, and the clotting time of each sample was obtained. The obtained clotting time was applied to the conversion formula mentioned above and converted to the fibrinogen concentration.

Fibrinogen concentration in sample=e^{7.2234}×(clotting time)^{-0.7636} [Formula 20]

The converted fibrinogen concentration was designated as the fibrinogen concentration in the citrated plasma sample.

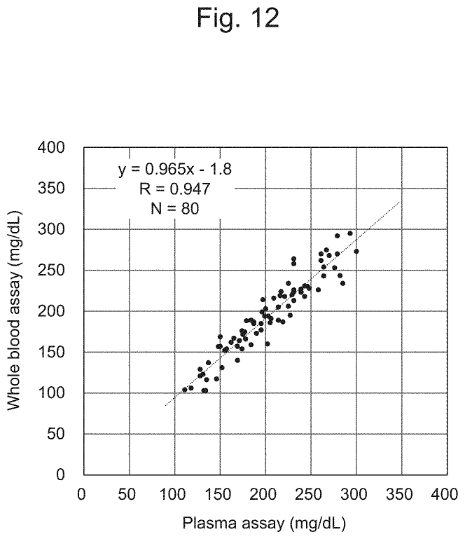

FIG. 12 shows the correlation between the quantified fibrinogen value determined using a citrated plasma sample and the quantified fibrinogen value determined using a citrated whole blood sample examined by the determination method disclosed herein. As is apparent from FIG. 12, the quantified fibrinogen value determined using a citrated whole blood sample is very consistent and highly correlated with the quantified fibrinogen value determined using a citrated plasma sample when the method disclosed herein is employed.

### Example 11

### Correlation Between the Measured Ht Value and the Peak Value of the Waveform

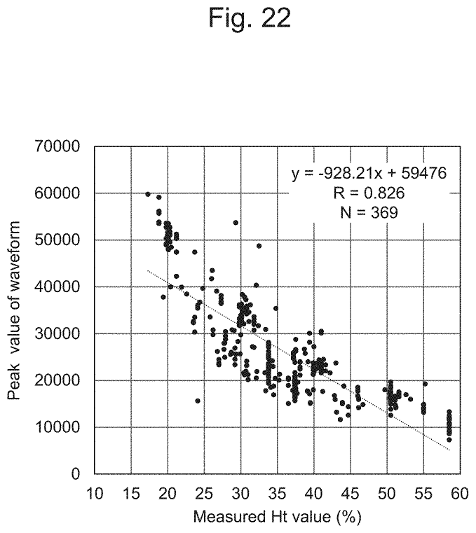

The measured Ht values and the peak values of the waveform were plotted to prepare a correlational chart (FIG. 22). Subsequently, the correlation coefficient and the linear regression formula were computed based on the correlational chart. As a result, a very strong correlation at R=0.826 was obtained. The approximation formula can be a formula other than the linear regression formula.

### Correlation Between the Measured Ht Value and the Waveform Ht Value

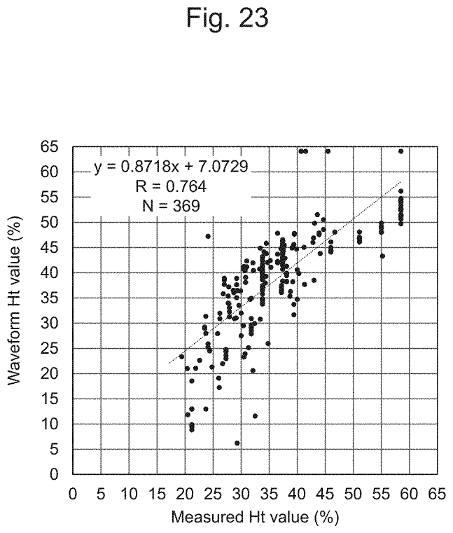

Subsequently, the waveform hematocrit value (the waveform Ht value) was computed based on the peak value of the waveform using the linear regression formula indicated above. Subsequently, the measured Ht values and the waveform Ht values were plotted to prepare a correlational chart (FIG. 23). Further, the correlation coefficient and the linear regression formula were computed based on the correlational chart. As a result, a strong correlation at R=0.764 was obtained. The approximation formula can be a formula other than the linear regression formula.

### Comparison Between the Fibrinogen Concentration in Plasma Determined by the Method of the Present Disclosure and the Fibrinogen Concentration in Plasma Determined by the Conventional Method

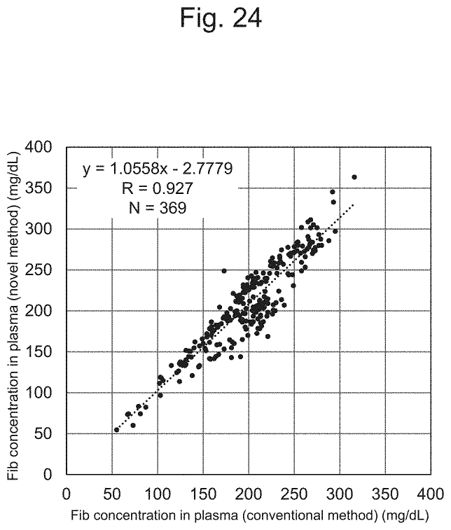

Subsequently, the fibrinogen concentration in whole blood was subjected to correction using the waveform Ht value above and the fibrinogen concentration in plasma (novel method) was computed. Subsequently, the fibrinogen concentration in whole blood was subjected to hematocrit correction using the hematocrit value measured using the blood cell counter MYTHIC22 (J) (A&T Corporation) to determine the fibrinogen concentration in plasma (conventional method). Subsequently, the fibrinogen concentration in plasma (conventional method) and the fibrinogen concentration in plasma (novel method) were plotted to prepare a correlational chart (FIG. 24). Further, the correlation coefficient and the linear regression formula were computed based on the correlational chart. As a result, a very strong correlation at R=0.927 was obtained. In this connection, the approximation formula can be a formula other than a linear regression formula. This indicates that measurement of the undiluted citrated whole blood sample using magnetic particles enables computation of the fibrinogen concentration in plasma without imputing hematocrit values via another means.

### INDUSTRIAL APPLICABILITY

According to the present disclosure, the fibrinogen concentration in plasma can be quantitatively measured without imputing the hematocrit values via another means.

All publications, patents, and patent applications cited herein are incorporated herein by reference in their entirety.

### DESCRIPTION OF REFERENCES

A: Transparent resin plate
B: Transparent resin plate
C: White resin plate
D: Reagent filling unit

1. A method for computing the fibrinogen concentration in plasma comprising:
(i) a step of adding a sample to a fibrinogen measurement dry reagent containing magnetic particles;
(ii) a step of allowing the magnetic particles in the reagent to move after the addition of the sample and monitoring the movement signal of the magnetic particles; and
(iii) a step of calculating a plurality of ratios of the movement signals of the magnetic particles monitored in step (ii) at a given time interval,
designating an arbitrary point within an interval during which the ratio of the movement signals of the magnetic particles calculated at a given time interval is maintained within a given range for a given period of time as the starting point, designating a point at or after the starting point at which the movement signal of the magnetic particles is attenuated by 5% to 50% from the peak value of the movement signal of the magnetic particles as the end point, designating the time from the starting point to the end point as the clotting time, and computing the fibrinogen concentration in whole blood based on the clotting time, and
computing a waveform-based hematocrit value based on the peak value of the movement signal of the magnetic particles, subjecting the computed fibrinogen concentration in whole blood to hematocrit correction using the waveform-based hematocrit value, and computing the fibrinogen concentration in plasma of the sample.

2. The method of claim 1, wherein the time interval used to compute the ratio of the movement signals of the magnetic particles is a given time interval selected from between 0.1 seconds and 2 seconds.

3. The method of claim 1, wherein the given range of the ratio of the movement signals of the magnetic particles is 1.0±0.2.

4. The method of claim 1, wherein the time period during which the ratio of the movement signals of the magnetic particles is maintained within a given range is 1.5 seconds.

5. The method of claim 1, wherein a point at or after the starting point at which the movement signal of the magnetic particles is attenuated by 20% to 30% from the peak value of the movement signal of the magnetic particles is designated as the end point.

6. The method of claim 1 comprising using a fibrinogen measurement dry reagent comprising:
(i) thrombin or a protein having thrombin activity;
(ii) magnetic particles;
(iii) a fibrin monomer polymerization inhibitor;
(iv) a calcium salt;
(v) a dry reagent layer solubility improving agent;
(vi) a dry reagent layer reinforcing material; and
(vii) a pH buffer.

7. A program for executing the method of claim 1.

8. An information recording medium comprising the program of claim 7 recorded thereon.

9. An apparatus for fibrinogen determination comprising the program of claim 7 integrated therein.

10. An apparatus for fibrinogen determination comprising the information recording medium of claim 8 stored therein.
